(19) United States
(12) Patent Application Publication
(10) Pub. No.: US 2024/0239884 A1
(43) Pub. Date: Jul. 18, 2024

(54) SOLUTION FORMULATIONS OF ENGINEERED ANTI-IL-23p19 ANTIBODIES

(71) Applicant: MERCK SHARP & DOHME LLC, Rahway, NJ (US)

(72) Inventors: Ramesh S. KASHI, Kenilworth, NJ (US); Aniket BADKAR, Portage, MI (US)

(21) Appl. No.: 18/405,772

(22) Filed: Jan. 5, 2024

Related U.S. Application Data
(63) Continuation of application No. 14/651,946, filed on Jun. 12, 2015.
(60) Provisional application No. 61/737,035, filed on Dec. 13, 2012.

(51) Int. Cl. C07K 16/24; A61K 39/395; A61K 39/00

(52) U.S. Cl. CPC C07K 16/244; A61K 39/39591; A61K 2039/505; C07K 2317/24; C07K 2317/51; C07K 2317/515; C07K 2317/56; C07K 2317/94

(57) ABSTRACT

The present invention provides high concentration solution formulations of anti-human interleukin-23 p19 (IL-23p19) antibody hum1388-b, and their use in treating various disorders.

### CROSS-REFERENCE TO RELATED APPLICATIONS

This application is a continuation of U.S. Application No. 14/651,946, filed Jun. 12, 2015, which is a national stage application under 35 U.S.C. § 371 of International Application No. PCT/US2013/073825, filed Dec. 9, 2013, which claims the benefit of U.S. Provisional Patent Application Ser. No. 61/737,035 filed on Dec. 13, 2012, the disclosures of which are explicitly incorporated by reference herein in their entireties.

### REFERENCE TO SEQUENCE LISTING

This application contains a Sequence Listing submitted as an electronic text file named “15-021-US-CON2_SequenceListing_ST26.xml,” having a size of 17,930 bytes, and created on Jan. 5, 2024. The information contained in this electronic file is hereby incorporated by reference in its entirety.

### FIELD OF THE INVENTION

The present invention relates generally to high concentration solution formulations of therapeutic antibodies, and their use in treating various disorders.

### BACKGROUND OF THE INVENTION

Interleukin-23 (IL-23) is a heterodimeric cytokine comprised of two subunits, p19 which is unique to IL-23, and p40, which is shared with interleukin-12 (IL-12). The p19 subunit is structurally related to IL-6, granulocyte-colony stimulating factor (G-CSF), and the p35 subunit of IL-12. IL-23 mediates signaling by binding to a heterodimeric receptor comprising two subunits, IL-23R, unique to IL-23 receptor, and IL-12Rβ1, which is shared with the IL-12 receptor. A number of early studies demonstrated that the consequences of a genetic deficiency in p40 (p40 knockout mouse; p40KO mouse) were more severe than those observed with deficiency of p35, e.g. in a p35KO mouse. These results were eventually explained by the discovery of IL-23, and the realization that the p40KO prevents expression of not only IL-12, but also IL-23. See, e.g., Oppmann et al. (2000) Immunity 13:715-725; Wiekowski et al. (2001) J. Immunol. 166:7563-7570; Parham et al. (2002) J. Immunol. 168:5699-708; Frucht (2002) Sci STKE 2002, E1-E3; Elkins et al. (2002) Infection Immunity 70:1936-1948).

Recent studies, through the use of p40 KO mice, have shown that blockade of both IL-23 and IL-12 is an effective treatment for various inflammatory and autoimmune disorders. However, the blockade of IL-12 through p40 appears to have undesirable systemic consequences, such as increased susceptibility to opportunistic microbial infections or increased risk of tumors. Bowman et al. (2006) Curr. Opin. Infect. Dis. 19:245; Langowski et al. (2006) Nature 442:461. Accordingly, specific blockade of the p19 subunit of IL-23 is preferred in the treatment of human disease because it interferes with the pathogenic inflammatory activity of IL-23 without interfering with the beneficial activities of IL-12. e.g. in fighting infection and in immunosurveillance.

Therapeutic antibodies may be used to block cytokine activity. A significant limitation in using antibodies as a therapeutic agent in vivo is the immunogenicity of the antibodies. For monoclonal antibodies derived from non-human species, repeated use in humans results in the generation of an immune response against the therapeutic antibody. Such an immune response results in a loss of therapeutic efficacy at a minimum, and potentially a fatal anaphylactic response. Accordingly, antibodies of reduced immunogenicity in humans, such as humanized or fully human antibodies, are preferred for treatment of human subjects. Exemplary therapeutic antibodies to IL-23p19 are disclosed in U.S. Patent Application Publication No. 2007/0009526, and in International Patent Publication Nos. WO 2007/076524, WO 2007/024846, WO 2007/147019, and WO 2009/043933 the disclosures of which are hereby incorporated by reference in their entireties. Additional humanized anti-IL-23p19 antibodies are disclosed in commonly assigned applications published as International Patent Publication Nos. WO 2008/103432 and WO 2008/103473, and in commonly-assigned U.S. Patent Application Publication No. 2007/0048315, the disclosures of which are hereby incorporated by reference in their entireties.

Antibody drugs for use in humans may differ somewhat in the amino acid sequence of their constant domains, or in their framework sequences within the variable domains, but they typically differ most dramatically in the CDR sequences. Even antibodies binding to the same protein, the same polypeptide, or even potentially the same epitope may comprise entirely different CDR sequences. Therapeutic antibodies for use in human beings can also be obtained from human germline antibody sequence or from non-human (e.g. rodent) germline antibody sequences, such as in humanized antibodies, leading to yet further diversity in potential CDR sequences. These sequence differences result in different stabilities in solution and different responsiveness to solution parameters. In addition, small changes in the arrangement of amino acids or changes in one or a few amino acid residues can result in dramatically different antibody stability and susceptibility to sequence-specific degradation pathways. As a consequence, it is not possible at present to predict the solution conditions necessary to optimize antibody stability. Each antibody much be studied individually to determine the optimum solution formulation. Bhambhani et al. (2012) J. Pharm. Sci. 101:1120.

Antibodies are also relatively high molecular weight proteins (˜150,000 Da), for example as compared with other therapeutic proteins such as hormones and cytokines. As a consequence, it is frequently necessary to dose with relatively high weight amounts of antibody drugs to achieve the desired molar concentrations of drug. In addition, it is often desirable to administer antibody drugs subcutaneously, as this enables self-administration. Self-administration avoids the time and expense associated with visits to a medical facility for administration, e.g., intravenously. Subcutaneous delivery is limited by the volume of solution that can be practically delivered at an injection site in a single injection, which is generally about 1 to 1.5 ml. Subcutaneous self-administration is typically accomplished using a pre-filled syringe or autoinjector filled with a liquid solution formulation of the drug, rather than a lyophilized form, to avoid the need for the patient to re-suspend the drug prior to injection. For delivery of higher doses of drug this volume limitation places a premium on the development of high concentration solution formulations. Such high concentrations of antibodies, however, exhibit macromolecular crowding effects and increased protein-protein interactions, resulting in physical instabilities such as opalescence, self-association, aggregation, unfolding and phase separation. Such high concentration antibody solutions can also exhibit high viscosity (e.g. >10 centipoises), which reduces syringeability in pre-filled syringes and autoinjector devices. Antibody drugs must be stable during storage to ensure efficacy and consistent dosing, so it is critical that whatever formulation is chosen supports desirable properties, such as high concentration, clarity and acceptable viscosity, and that is also maintains these properties and drug efficacy over an acceptably long shelf-life under typical storage conditions.

As a consequence, the need exists for stable, high concentration solution formulations of therapeutic antibodies, such as antibodies that bind to human IL-23p19. Such stable solution formulations will preferably exhibit stability over months to years under conditions typical for storage of drugs for self-administration, i.e. at refrigerator temperature in a syringe, resulting in a long shelf-life for the corresponding drug product. Such stable, high-concentration solution formulations would enable packaging of the antibody drug for high concentration subcutaneous injection by self-administration.

### SUMMARY OF THE INVENTION

The present invention provides high concentration solution formulations of humanized anti-IL-23p19 antibody 13B8-b (“hum13B8-b”). Antibody hum13B8-b comprises two identical light chains with the sequence of SEQ ID NO: 2 and two identical heavy chains with the sequence of SEQ ID NO: 1.

In one embodiment, the solution formulation comprises humanized anti-IL-23p19 antibody hum13B8-b, histidine buffer pH 6.0 (±0.3), sucrose and polysorbate 80. In anther embodiment, the solution formulation comprises humanized anti-IL-23p19 antibody 13B8-b, about 10 mM histidine buffer pH 6.0 (±0.3), about 7% sucrose and about 0.05% polysorbate 80. In a further embodiment, the solution formulation comprises humanized anti-IL-23p19 antibody 13B8-b, 10 mM histidine buffer pH 6.0 (±0.3), 7% sucrose and 0.05% polysorbate 80.

In various embodiments, the solution formulations of the present invention comprise at least 50, 80, 90, 100, 110 or 120 mg/ml antibody hum13B8-b. In other embodiments, the solution formulations of the present invention comprise about 80-120 mg/ml antibody hum13B8-b, 80 -120 mg/ml antibody hum13B8-b, about 100 mg/ml antibody hum13B8-b, and 100 mg/ml antibody hum13B8-b.

In another aspect the invention relates to methods of treatment employing the high concentration solution formulations of anti-IL-23p19 antibody hum13B8-b of the present invention to treat disorders including, but not limited to, inflammatory disease, autoimmune disease, proliferative disorders, cancer, infectious disease (e.g. bacterial, mycobacterial, viral or fungal infection, including chronic infections), arthritis, psoriasis, psoriatic arthritis, enthesitis, ankylosing spondlyitis, inflammatory bowel disease, including Crohn's disease and ulcerative colitis, multiple sclerosis, uveitis, graft-versus-host disease, systemic lupus erythematosus and diabetes. In yet another aspect the invention relates to high concentration solution formulations of anti-IL-23p19 antibody hum13B8-b for use in treating these same disorders. In yet another aspect the invention relates to use of high concentration solution formulations of anti-IL-23p19 antibody hum13B8-b in manufacture of a medicament for use in treating these same disorders.

### BRIEF DESCRIPTION OF THE DRAWINGS

FIGS. 1A-1K show data used to determine optimal buffer and pH conditions for formulations comprising hum13B8-b. FIG. 1A shows unfolding temperature, as determined by differential scanning calorimetry (DSC), and percent purity, as measured by RP-HPLC, for various 1 mg/mL antibody formulations, and FIG. 1B shows percent monomer and percent late-eluting peaks, as measured by HP-SEC, as a function of pH for several different buffers (acetate, citrate, phosphate and Tris). Brief discussions of DSC and HP-SEC are provided Examples 2 and 3, respectively.

FIGS. 1C-1E show various properties of 10 mM citrate formulations as a function of pH, including opalescence (OD₃₅₀), hydrodynamic size (nm) distribution, as measured by dynamic light scattering (DLS), and melting temperature, as determined by DSC, respectively. Brief discussions of DLS and DSC are provided Examples 4 and 2, respectively.

FIGS. 1F and 1G show percentage of late eluting peaks, and percentage of main peak, respectively, over time, as determined by high performance ion exchange chromatography (HP-IEX). A brief discussion of HP-IEX is provided Example 5.

FIGS. 1H and 1I show opalescence (OD₃₅₀) and hydrodynamic size (nm), as measured by dynamic light scattering (DLS), respectively, for low concentration antibody formulations (1 mg/ml) in several buffers at different pHs, whereas FIG. 1J shows the hydrodynamic size (nm) distribution for more concentrated antibody formulations (50 and 100 mg/ml), as determined by DLS. A brief discussion of DLS is provided Example 4.

FIG. 1K shows the percentage of late eluting peaks in various buffers at different pHs over time, as determined by HP-SEC. A brief discussion of HP-SEC is provided Example 3.

FIGS. 2A and 2B show the unfolding temperature, as determined by DSC, and changes in opalescence, respectively, for formulations of hum13B8-b containing various excipients (100 mM NaCl, 7% sucrose, 7% trehalose and 6% mannitol). DS refers to drug substance. A brief discussion of DSC is provided Example 2.

FIGS. 3A and 3B show percent antibody and percent early eluting peaks, as determined by HP-SEC, respectively, and FIG. 3C shows opalescence, before and after five days of shaking, as a function of the presence or absence of surfactant (0.05% polysorbate 20, 0.05% polysorbate 80, or Pluronic F-68). The 5 Day Shake value for the no surfactant (NS) sample is actually over 3, and thus well off-scale. A brief discussion of HP-SEC is provided Example 3

FIGS. 4A-4M show stability of acetate and histidine antibody formulations comprising 10 mM buffer, 7% sucrose and 0.05% polysorbate 80, when stored under various conditions. Samples were stored as 1.5 ml samples in 2.0 ml glass vials. FIG. 4A shows stability of 50 and 100 mg/ml antibody preparations, as reflected by percentage of monomer antibody measured by HP-SEC, when stored at 5° C.(ambient relative humidity) or under RH4 conditions (40° C., 75% relative humidity). A brief discussion of HP-SEC is provided Example 3.

FIGS. 4B-4D are plots of the percent monomer, HMW species and LMW species, respectively, as determined by HP-SEC, under a variety of storage conditions “25H” refers to storage at 25° C., 60% relative humidity. Note that the ordinates (time axes) of FIGS. 4B-4D are not linear. A brief discussion of HP-SEC is provided Example 3.

FIGS. 4E and 4F are plots of results of HP-IEX experiments that monitor antibody stability by measuring percentages of the main peak and acidic variants, respectively. A brief discussion of HP-IEX is provided Example 5.

FIGS. 4G-4I are plots of opalescence after storage at 5° C., 25° C. (25H) and 40° C. (RH4), respectively. FIGS. 4J and 4K show oxidation, as measured by peptide mapping, after storage at RH4 (40° C., 75% relative humidity) and 5° C., and at 40° C. and 5° C., respectively. A brief discussion of peptide mapping is provided Example 6.

FIGS. 4L and 4M are unfolding plots from DSC experiments on acetate and histidine formulations, respectively, for initial samples and samples stored 4.5 months at 5° C. or RH4 conditions. A brief discussion of DSC is provided Example 2.

The stabilities of 10 mM acetate and 10 mM histidine formulations were measured as percent monomer and as percent high molecular weight species, as measured by HP-SEC. Stability was measured for samples stored at 5° C. (±3° C., 25H (25° C., 60% relative humidity), or RH4 (40° C., 75% relative humidity). Results are presented at FIGS. 4A and 4B. A brief discussion of HP-SEC is provided Example 3.

FIGS. 5A-5L present stability data for antibody formulations of the present invention when stored as drug substance in 30 mL ethylene-vinyl-acetate (EVA) fluid contact layer Celsius® Pak bags. Data are presented for formulations comprising 10 mM Histidine buffer (pH 6.0), 7% sucrose, 0.05% polysorbate 80 and antibody hum13B8-b. FIGS. 5A-5C are plots of protein concentration, biological potency (as measured by cell based functional assay), and biological potency (as measured by ELISA), respectively, for three different preparations of hum13B8-b (Lots A, B and C). Brief discussions of protein concentration determination, cell based functional assays and ELISAs are provided Examples 7, 8 and 9, respectively.

FIGS. 5D-5G are plots of results of HP-IEX experiments that monitor antibody stability by measuring percentages of acidic variants, main peak, post-main peak species and basic variants, respectively. A brief discussion of HP-IEX is provided Example 5.

FIG. 5H shows the percent monomer, as measured by HP-SEC. A brief discussion of HP-SEC is provided Example 3.

FIGS. 5I and 5J show purity by measuring percent main peak by non-reducing CE-SDS, or percent heavy and light chains by reducing CE-SDS, respectively. A brief discussion of CE-SDS is provided Example 10.

FIGS. 5K and 5L show percent HMW and LMW species, respectively, as measured by HP-SEC. A brief discussion of HP-SEC is provided Example 3.

FIGS. 6A-6K present additional stability data for antibody formulations of the present invention (antibody Lots A and B) when stored as drug substance in 30 mL ethylene-vinyl-acetate (EVA) fluid contact layer Celsius Pak bags. Data are presented for formulations comprising 10 mM Histidine buffer (pH 6.0), 7% sucrose, 0.05% polysorbate 80 and antibody hum13B8-b. FIGS. 6A and 6B are plots of biological potency as measured by ELISA and as measured by cell based functional assay, respectively. Brief discussions of ELISAs and cell based functional assays are provided Examples 9 and 8, respectively.

FIGS. 6C-6E are plots of the percent monomer, HMW species and LMW species, respectively, as determined by HP-SEC, under a variety of storage conditions. A brief discussion of HP-SEC is provided Example 3.

FIGS. 6F and 6G show purity by measuring percent main peak by non-reducing CE-SDS, or percent heavy and light chains by reducing CE-SDS, respectively. A brief discussion of CE-SDS is provided Example 10.

FIGS. 6H-6K are plots of results of HP-IEX experiments that monitor antibody stability by measuring percentages of acidic variants, main peak, post-main peak species and basic variants, respectively. A brief discussion of HP-IEX is provided Example 5.

FIGS. 7A-7L present stability data for antibody formulations of the present invention when stored at 5° C. (3° C.) as drug product in unit doses in prefilled syringes, at 100 mg/ml antibody concentration and 1.0 ml fill volume. FIG. 7A is a plot of protein concentration for four different preparations of hum13B8-b (Lots D, E, F, G and H) as determined by uv absorption. A brief discussion of protein concentration determinations is provided Example 7.

FIGS. 7B and 7C are plots of biological potency as measured by cell based functional assay, and as measured by ELISA, respectively, for four different preparations of hum13B8-b (Lots E, F, G and H). Brief discussions of cell based functional assays and ELISAs are provided Examples 8 and 9, respectively.

FIGS. 7D-7F are plots of the percent HMW species, monomer, and LMW species, respectively, as determined by HP-SEC. A brief discussion of HP-SEC is provided Example 3.

FIGS. 7G-7J are plots of results of HP-IEX experiments that monitor antibody stability by measuring percentages of the main peak, acidic variants, basic variants, and post-main peak species, respectively. A brief discussion of HP-IEX is provided Example 5.

FIGS. 7K and 7L show purity by measuring percent main peak by non-reducing CE-SDS, or percent heavy and light chains by reducing CE-SDS, respectively. A brief discussion of CE-SDS is provided Example 10.

### DETAILED DESCRIPTION

As used herein, including the appended claims, the singular forms of words such as “a,” “an,” and “the,” include their corresponding plural references unless the context clearly dictates otherwise. Table 10 below provides a listing of sequence identifiers used in this application. Unless otherwise indicated, the proteins and subjects referred to herein are human proteins and human subjects, rather than another species. As used herein, “FIG. X” refers collectively to all of individual FIGS. XA-XZ.

All references cited herein are incorporated by reference to the same extent as if each individual publication, database entry (e.g. Genbank sequences or GeneID entries), patent application, or patent, was specifically and individually indicated to be incorporated by reference. Citation of the references herein is not intended as an admission that the reference is pertinent prior art, nor does it constitute any admission as to the contents or date of these publications or documents.

### I. Definitions

“Proliferative activity” encompasses an activity that promotes, that is necessary for, or that is specifically associated with, e.g., normal cell division, as well as cancer, tumors, dysplasia, cell transformation, metastasis, and angiogenesis.

As used herein, the term “hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196: 901-917). As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

“Inflammatory disorder” means a disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

As used herein, concentrations are to be construed as approximate within the ranges normally associated with such concentrations in the manufacture of pharmaceutical formulations. Specifically, concentrations are need not be exact, but may differ from the stated concentrations within the tolerances typically expected for drugs manufactured under GMP conditions. Similarly, pH values are approximate within the tolerances typically expected for drugs manufactured under GMP conditions and stored under typical storage conditions. For example, the histidine formulations of the present invention are referred to as having a pH of 6.0 but the typical tolerance is pH 6.0 (±0.3). Unless otherwise indicated, percent concentrations are weight/weight concentrations.

### II. High Concentration Solution Antibody Formulations

Typical therapeutic monoclonal antibodies are comprised of four polypeptides: two light chains (e.g. 214 amino acids long) and two heavy chains (e.g. 446 amino acids long). Each chain is in turn comprised of a variable domain and a constant domain. Variable domains for anti-IL-23p19 hum13B8-b are 108 and 116 amino acids for the light and heavy chains, respectively, and the constant domains are 106 and 330 amino acids. The specificity of an antibody for its target is largely determined by the sequences falling within the so-called “hyper-variable,” or “complementarity determining” regions (CDRs), three of which are found in the variable domains of each of the heavy and light chains. The CDRs may vary in length between different antibodies, but in hum13B8-b the CDRs comprise 44 amino acids on heavy chains and 27 amino acids on the light chain. The CDR residues are highly variable between different antibodies, and may originate from human germline sequences (in the case of fully human antibodies), or from non-human (e.g. rodent) germline sequences. The framework regions can also differ significantly from antibody to antibody. The constant regions will differ depending on whether the selected antibody has a lambda (λ) or kappa (κ) light chain, and depending on the class (or isotype) of the antibody (IgA, IgD, IgE, IgG, or IgM) and subclass (e.g. IgG1, IgG2, IgG3, IgG4). The sum total is an antibody molecule of approximately 150,000 Da, comprised of approximately 650 amino acids, of which 224 are in variable domains, including 71 amino acids in “hyper-variable” regions, with constant domains varying in class, subclass, and light chain constant domains.

The antibody of the present invention (anti-IL-23p19 mAb hum13B8-b) also differs from many recently developed therapeutic antibodies in that it is humanized, rather than fully human. As a result, the CDR sequences are derived from non-human (in this case mouse) germline sequences, rather than human germline sequences. The germline sequences comprise the sequence repertoire from which an antibody's CDR sequences are derived, aside from somatic hypermutation derived changes, and as a consequence it would be expected that CDRs obtained starting with a mouse germline would systematically differ from those starting from a human germline. This is, in fact, the basis for using different species immune systems to raise antibodies, since use of different species increases the potential diversity in resulting CDR sequences. Use of human germline sequences is often justified on the basis that CDR sequences from human germlines will be less immunogenic in humans than those derived from other species, reflecting the underlying belief that CDRs will systematically differ depending on their species of origin. Although the increase in CDR diversity increases the likelihood of finding antibodies with desired properties, such as high affinity, it further magnifies the difficulties in developing a stable solution formulation of the resulting antibody.

Even antibodies that bind to the same antigen can differ dramatically in sequence, and are not necessarily any more closely related in sequence than antibodies to entirely separate antigens. For example, the variable domains of the antibody of the present invention (hum13B8-b) share only approximately 50 to 60% sequence identity with another anti-IL-23p19-specific antibody CNTO 1959 (SEQ ID NOs: 116 and 106 of U.S. Pat. No. 7,993,645). CNTO 1959 is a fully human antibody. Based on the low sequence similarity, the chemical properties of the antibodies, and thus their susceptibility to degradation, cannot be presumed to be similar despite their shared target.

As demonstrated above, antibodies are large, highly complex polypeptide complexes subject to various forms of degradation and instability in solution. The diversity of sequence, and thus structure, of antibodies gives rise to wide range of chemical properties. Aside from the obvious sequence-specific differences in antigen binding specificity, antibodies exhibit varying susceptibility to various degradative pathways, aggregation, and precipitation. Amino acid side chains differ in the presence or absence of reactive groups, such as carboxy- (D,E), amino- (K), amide- (N,Q), hydroxyl- (S,T,Y), sulfhydryl- (C), thioether- (M) groups, as well as potentially chemically reactive sites on histidine, phenylalanine and proline residues. Amino acid side chains directly involved in antigen binding interactions are obvious candidates for inactivation by side chain modification, but degradation at other positions can also affect such factors as steric orientation of the CDRs (e.g. changes in framework residues), effector function (e.g. changes in Fc region—see, e.g., Liu et al. (2008) Biochemistry 47:5088), or self-association/aggregation.

Antibodies are subject to any number of potential degradation pathways. Oxidation of methionione residues in antibodies, particularly in CDRs, can be a problem if it disrupts antigen binding. Presta (2005) J. Allergy Clin. Immunol. 116: 731; Lam et al. (1997) J. Pharm. Sci. 86:1250. Other potential degradative pathways include asparagine deamidation (Harris et al. (2001) Chromatogr., B 752:233; Vlasak et al. (2009) Anal. Biochem. 392:145) tryptophan oxidation (Wei et al. (2007) Anal. Chem. 79:2797), cysteinylation (Banks et al. (2008) J. Pharm. Sci. 97:775), glycation (Brady et al. (2007) Anal. Chem. 79:9403), pyroglutamate formation (Yu et al. (2006) J. Pharm. Biomed. Anal. 42:455), disulfide shuffling (Liu et al. (2008) J. Biol. Chem. 283:29266), and hydrolysis (Davagnino et al. (1995) J. Immunol. Methods 185:177). Discussed in Ionescu & Vlasak (2010) Anal. Chem. 82:3198. See also Liu et al. (2008) J. Pharm. Sci. 97:2426. Some potential degradation pathways depend not only on the presence of a specific amino acid residue, but also the surrounding sequence. Deamidation and isoaspartate formation can arise from a spontaneous intramolecular rearrangement of the peptide bond following (C-terminal to) N or D residues, with N-G and D-G sequences being particularly susceptible. Reissner & Aswad (2003) CMLS Cell. Mol. Life Sci. 60:1281.

Antibodies are also subject to sequence-dependent non-enzymatic fragmentation during storage. Vlasak & Ionescu (2011) mAbs 3:253. The presence of reactive side chains, such as D, G. S. T, C or N can result in intramolecular cleavage reactions that sever the polypeptide backbone. Such sequence specific hydrolysis reactions are typically critically dependent on pH. Id. Antibodies may also undergo sequence-dependent aggregation, for example when CDRs include high numbers of hydrophobic residues. Perchiacca et al. (2012) Prot. Eng. Des. Selection 25:591. Aggregation is particularly problematic for antibodies that need to be formulated at high concentrations for subcutaneous administration, and has even led some to modify the antibody sequence by adding charged residues to increase solubility. Id.

Mirroring the diversity of potential sequence-specific stability issues with antibodies, potential antibody formulations are also diverse. A number of different variables must be custom-optimized for each new antibody. Formulations may vary, for example, in antibody concentration, buffer, pH, presence or absence of surfactant, presence or absence of tonicifying agents (ionic or nonionic), presence of absence of molecular crowding agent. Commercially available therapeutic antibodies are marketed in a wide range of solution formulations, in phosphate buffer (e.g. adalimumab), phosphate/glycine buffer (e.g. basilixumab), Tris buffer (e.g. ipilimumab), histidine (e.g. ustekinumab), sodium citrate (e.g. rituximab); and from pH 4.7 (e.g. certolizumab) and pH 5.2 (e.g. adalimumab) to pH 7.0-7.4 (e.g. cetuximab). They are also available in formulations optionally containing disodium edetate (e.g. alemtuzumab), mannitol (e.g. ipilimumab), sorbitol (e.g. golimumab), sucrose (e.g. ustekinumab), sodium chloride (e.g. rituximab), potassium chloride (e.g. alemtuzumab), and trehalose (e.g. ranibizumab); all with and without polysorbate-80, ranging from 0.001% (e.g. abcixmab) to 0.1% (e.g. adalimumab).

Exemplary antibody formulations are found at U.S. Pat. No. 7,691,379 (anti-IL-9 mAb MEDI-528); U.S. Pat. No. 7,592,004 (anti-IL-2 receptor, daclizumab); U.S. Pat. No. 7,705,132 (anti-EGFR, panitumumab); and U.S. Pat. No. 7,635,473 (anti-Aβ; bapineuzumab). Additional exemplary antibody formulations are found at U.S. Pat. App. Pub. Nos. 2010/00021461 (anti-α4-integrin, natalizumab); 2009/0181027 (anti-IL-12/IL-23, ustekinumab); 2009/0162352 (anti-CD20, ritumixmab); 2009/0060906 (anti-IL-13); 2008/0286270 (anti-RSV, palivizumab); and 2006/0088523 (anti-Her2, pertuzumab). Yet additional formulations are described at Daugherty & Mrsyn (2006) Adv. Drug Deliv. Rev. 58:686; Wang et al. (2007) J. Pharm. Sci. 96:1; and Lam et al. (1997) J. Pharm. Sci. 86:1250.

Sequence variability, which is the basis for antibody specificity, is at the heart of the immune response. This variability leads to chemical heterogeneity of the resulting antibodies, which results in a wide range of potential degradation pathways. The vast array of antibody formulations developed to-date attests to the fact that formulations must be individually optimized for each specific antibody to ensure optimal stability. In fact, each and every commercial therapeutic antibody approved for use in humans so far has had a unique, distinct formulation.

### III. Biological Activity of Humanized Anti-IL-23

The solution formulations of anti-IL-23p19 mAb hum13B8-b of the present invention will find use in treatment of disorders in which selective antagonism of IL-23 signaling is expected to be beneficial. Inflammatory diseases of the skin, joints, CNS, as well as proliferative disorders elicit similar immune responses, thus IL-23 blockade should provide inhibition of these immune mediated inflammatory disorders, without comprising the host ability to fight systemic infections. Antagonizing IL-23 should relieve the inflammation associated with inflammatory bowel disease, Crohn's disease, ulcerative colitis, rheumatoid arthritis, psoriatic arthritis, psoriasis, ankylosing spondylitis, graft-versus-host disease, atopic dermatitis, and various other autoimmune and inflammatory disorders. Use of IL-23 inhibitors will also provide inhibition of proliferative disorders, e.g., cancer and autoimmune disorders, e.g., multiple sclerosis, type I diabetes, and SLE. Descriptions of IL-23 in these various disorders can be found in the following published PCT applications: WO 04/081190; WO 04/071517; WO 00/53631; and WO 01/18051. IL-23 inhibitors may also find use in treatment of infections, including chronic infections, such as bacterial, mycobacterial, viral and fungal infections. See U.S. Pat. No. 8,263,080 and Int'l App. Pub. WO 2008/153610.

The high concentration solution formulations of the present invention include antibodies that retain biologically activity when stored for extended periods of time. As used herein, the term “biologically active” refers to an antibody or antibody fragment that is capable of binding the desired the antigenic epitope and directly or indirectly exerting a biologic effect. Typically, these effects result from the failure of IL-23 to bind its receptor. As used herein, the term “specific” refers to the selective binding of the antibody to the target antigen epitope. Antibodies can be tested for specificity of binding by comparing binding of the antibody to IL-23 with binding to irrelevant antigen or antigen mixture under a given set of conditions. If the antibody binds to IL-23 at least 10, and preferably 50 times more than to irrelevant antigen or antigen mixture then it is considered to be specific. An antibody that binds to IL-12 is not an IL-23-specific antibody. An antibody that “specifically binds” to IL-23p19 does not bind to proteins that do not comprise the IL-23p19-derived sequences, i.e. “specificity” as used herein relates to IL-23p19 specificity, and not any other sequences that may be present in the protein in question. For example, as used herein, an antibody that “specifically binds” to IL-23p19 will typically bind to FLAG®-hIL-23p19, which is a fusion protein comprising IL-23p19 and a FLAG® peptide tag, but it does not bind to the FLAG® peptide tag alone or when it is fused to a protein other than IL-23p19.

IL-23-specific binding compounds of the present invention can inhibit any of its biological activities, including but not limited to production of IL-1β and TNF by peritoneal macrophages and IL-17 by T_{H}17 T cells. See Langrish et al. (2004) Immunol. Rev. 202:96-105. Anti-IL-23p19 antibodies will also be able to inhibit the gene expression of IL-17A, IL-17F, CCL7, CCL17, CCL20, CCL22, CCR1, and GM-CSF. See Langrish et al. (2005) J. Exp. Med. 201:233-240. IL-23-specific binding compounds of the present invention will also block the ability of IL-23 to enhance proliferation or survival of TH17 cells. Cua and Kastelein (2006) Nat. Immunol. 7:557-559. The inhibitory activity of engineered anti-IL-23p19 antibodies will be useful in the treatment of inflammatory, autoimmune, and proliferative disorders. Examples of such disorders are described in PCT patent application publications WO 04/081190; WO 04/071517; WO 00/53631; and WO 01/18051.

The high concentration solution formulations of the present invention are useful, for example, for storage and delivery of anti-IL-23p19 antibody hum13B8-b for use in treatment or prevention of a disorder associated with elevated activity of IL-23 or IL-23p19, such as Th17-mediated diseases, autoimmune or chronic inflammatory disorders, or cancers.

### IV. Solution Formulations of Humanized Anti-IL-23p19 Antibody 13B8-b

The present invention provides high concentration solution formulations of anti-IL-23p19 antibody hum13B8-b, which comprises two identical light chains with the sequence of SEQ ID NO: 2 and two identical heavy chains with the sequence of SEQ ID NO: 1, and which is disclosed in co-pending, commonly assigned U.S. Pat. No. 8,293,883, the disclosure of which is hereby incorporated by reference in its entirety. The humanized light chain 13B8 sequence (with kappa constant region) is provided at SEQ ID NO: 2, and the light chain variable domain comprises residues 1-108 of that sequence. The humanized heavy chain 13B8 sequence (with γ1 constant region) is provided at SEQ ID NO: 1, and the heavy chain variable domain comprises residues 1-116 of that sequence.

Heavy and light chain sequences (SEQ ID NOs: 1 and 2) are provided without signal sequences. Exemplary heavy and light chain signal sequences are provided at SEQ ID NOs: 12 and 13, respectively. The signal sequences, or nucleic acid sequences encoding the signal sequences, may be appended to the N-terminus of the respective antibody chains to create a precursor protein for secretion from a host cell. Alternative signal sequences may also be used, and several can be found at “SPdb: a Signal Peptide Database.” Choo et al. (2005) BMC Bioinformatics 6:249.

A hybridoma expressing parental antibody 13B8 was deposited pursuant to the Budapest Treaty with American Type Culture Collection (ATCC—Manassas, Virginia, USA) on Aug. 17, 2006 under Accession Number PTA-7803. The relationship between parental antibody 13B8 and hum13B8-b is detailed in commonly assigned U.S. Pat. No. 8,293,883.

Solution formulations of the present invention were developed using at least eight different lots of antibody 13B8-b prepared from Chinese hamster ovary (CHO) cells in culture, at 500-2000 L scale.

A range of initial solution conditions was considered for the solution formulation of the present invention. Experiments were done with various buffers, such as acetate, citrate, histidine, TRIS and phosphate, at pHs ranging from 4.0 to 8.8. Excipients, such as sucrose, trehalose, and mannitol, were tested, as were various concentrations (and thus ionic strengths) of NaCl, and the inclusion of the surfactant polysorbate 80. Formulations were screened based on opalescence by determining the O.D. at 350 nm. Aggregation was measured by high-performance size exclusion chromatography (HP-SEC), dynamic light scattering (DLS) and analytical ultracentrifugation (AUC). Biochemical stability was measured by high-performance ion exchange chromatography (HP-IEX), and thermal stability was measured by differential scanning calorimetry (DSC).

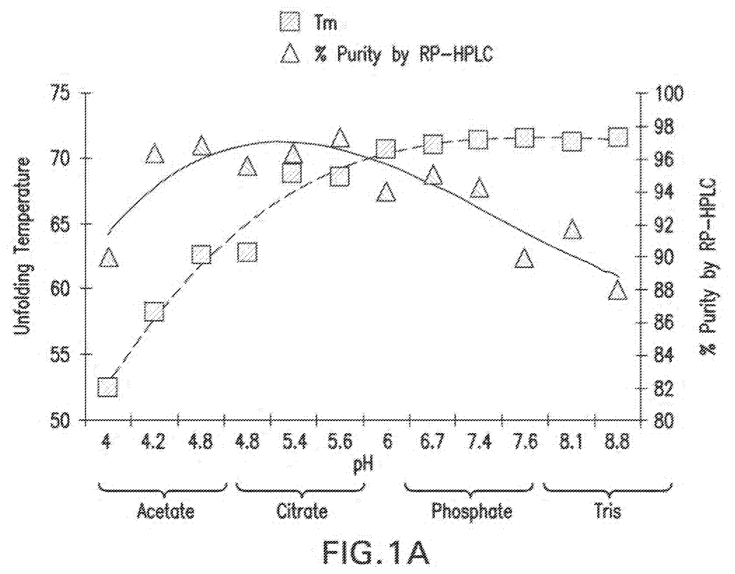

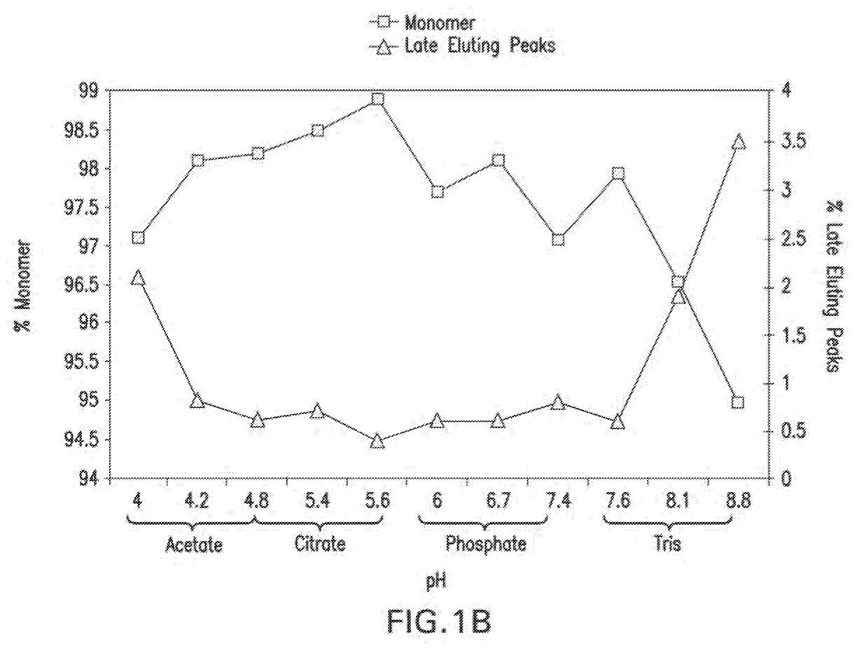

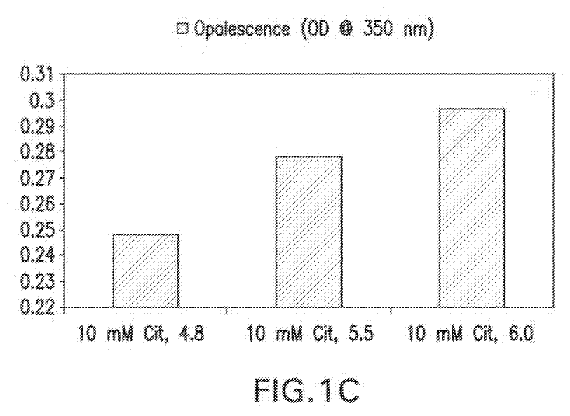

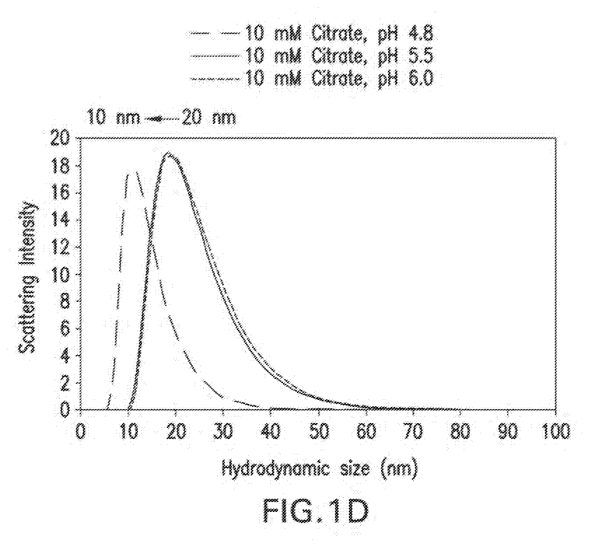

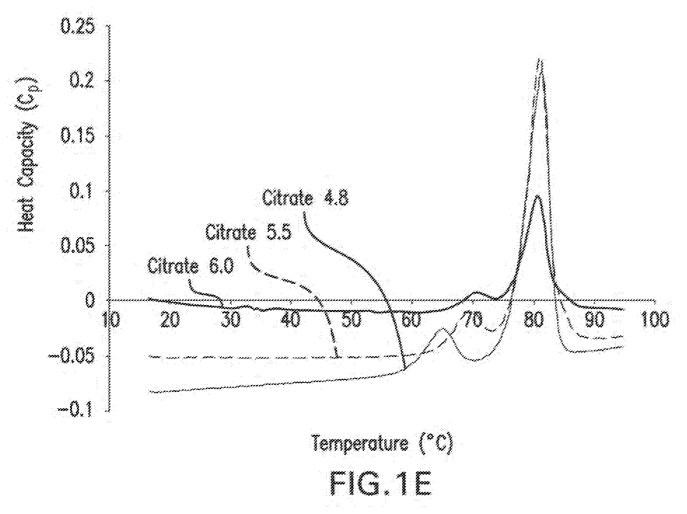

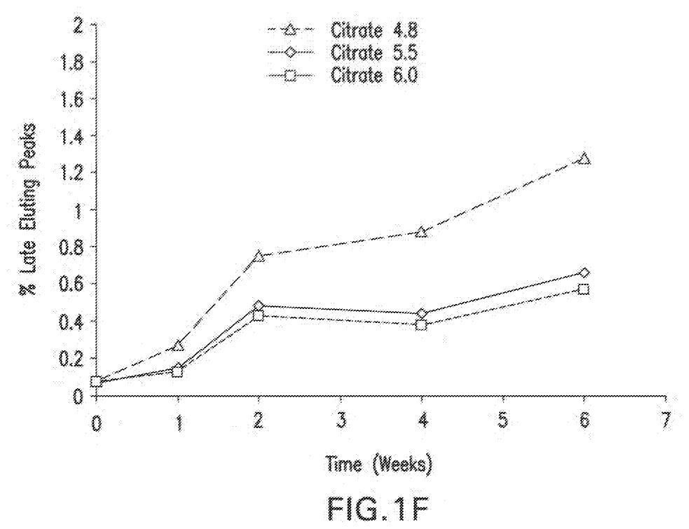

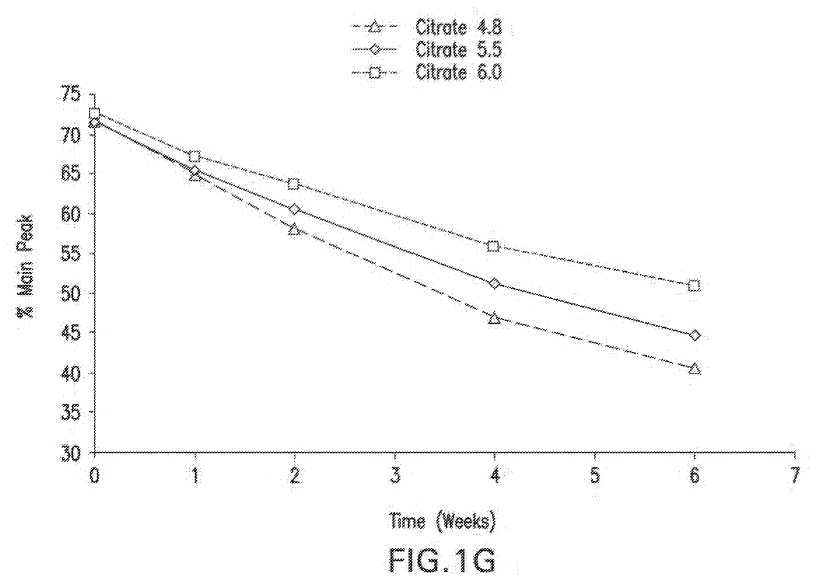

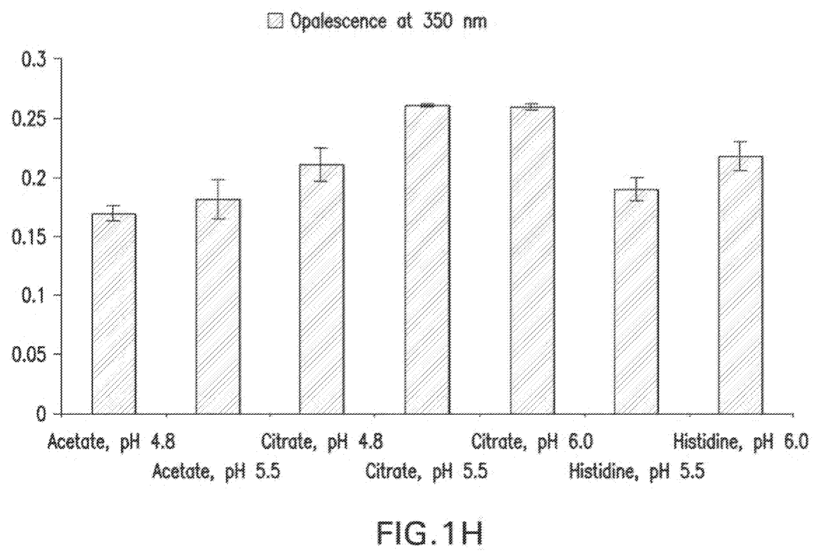

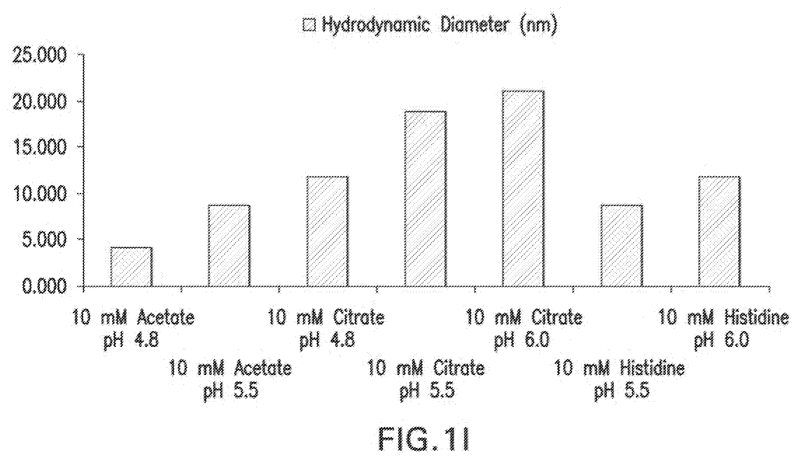

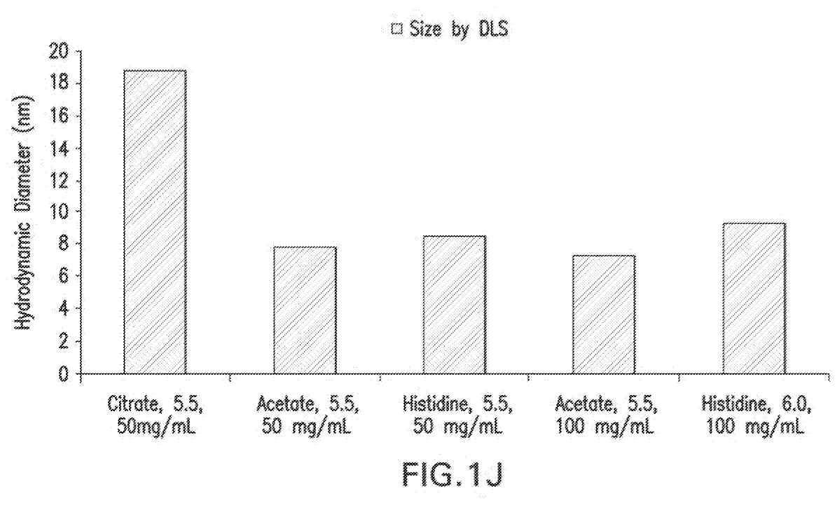

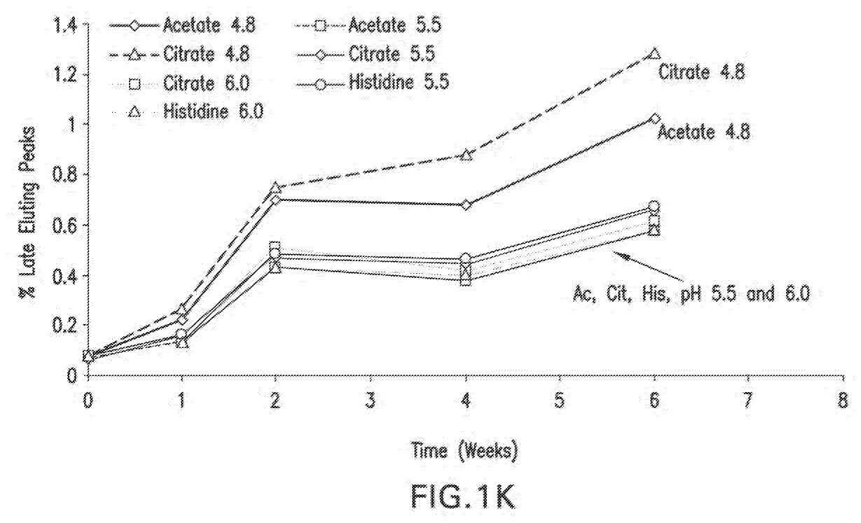

Initial pre-formulation experiments at 1 mg/mL hum13B8-b revealed that the percent purity, as measured by reverse phase high performance liquid chromatography (RP-HPLC), increased up to about pH 6.0 and then remained steady up to pH 8.8. See FIG. 1A. However, the unfolding temperature of the antibody peaked around pH 5. See FIG. 1A. Comparison of buffer species between acetate, citrate, phosphate and TRIS showed that citrate buffer at pH 5.5 gave the highest percentage of monomer, and the lowest percentage of late-eluting peaks. See FIG. 1B. Although optimal biochemical and biophysical stability were observed in citrate buffer at pH 5.5, concentration of antibody in citrate buffer to >65 mg/mL antibody gave rise to substantial opalescence (data not shown). Opalescence is undesirable due to the potential for decreased patient acceptance, which is of particular concern for a drug that may be formulated for self-administration. Although this opalescence was reversible upon dilution and lowering the pH to 4.8 in citrate buffer (FIG. 1C), and lowering the pH also lowered the hydrodynamic diameter (FIG. 1D), lowering the pH also decreased the thermal and biochemical stability of the solution, as reflected by an increased proportion of low-melting forms (FIG. 1E), increased accumulation of late-eluting peaks over time, as measured by HP-SEC (FIG. 1F), and a decrease in main peak over time, as measured by HP-IEX (FIG. 1G).

Histidine was then considered as an alternative buffer system in an attempt to decrease opalescence and self-association without decreasing thermal and biochemical stability. Samples were prepared in 10 mM acetate (pH 4.8 and 5.6), 10 mM citrate (pH 4.8, 5.5 and 6.0), and 10 mM histidine (pH 5.5 and 6.0). Opalescence and hydrodynamic diameter were determined by OD₃₅₀ and DLS, respectively. See FIGS. 1H and 1I. Replacement of citrate with either acetate or histidine minimized opalescence and decreased self-association without compromising biochemical stability. At higher antibody concentrations (50 to 100 mg/mL), acetate (pH 5.5) and histidine (pH 6.0) formulations were clear and did not have the increased hydrodynamic size that had been observed with citrate (pH 5.5). See FIG. 1J. Low pH (4.8) citrate and acetate formulations also led to increased accumulation of late-eluting peaks during storage under RH4 conditions (40° C., 75% relative humidity), as measured by size exclusion chromatography (SEC). See FIG. 1K.

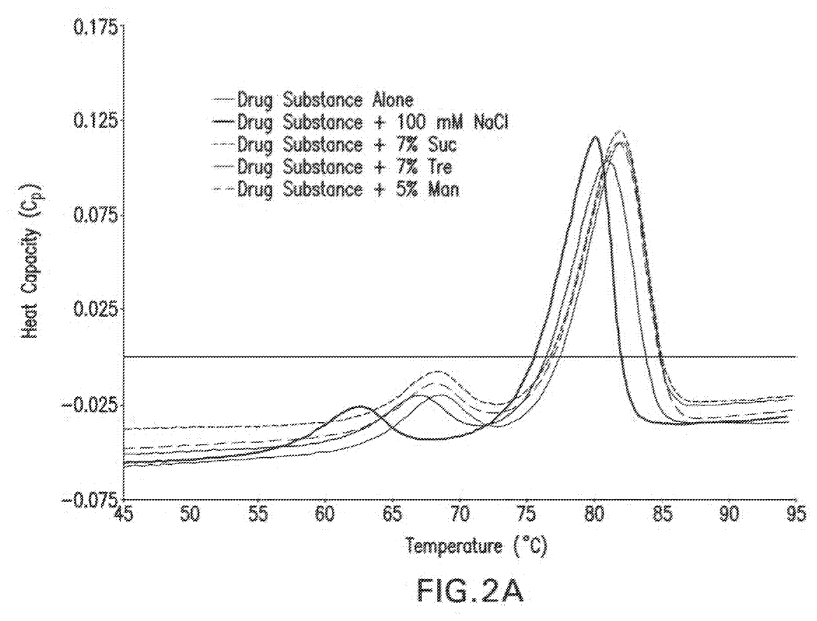

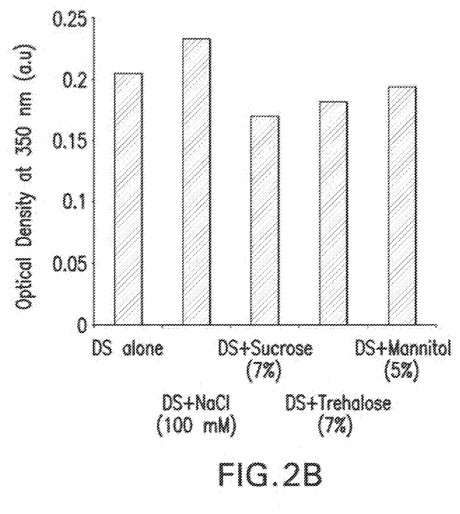

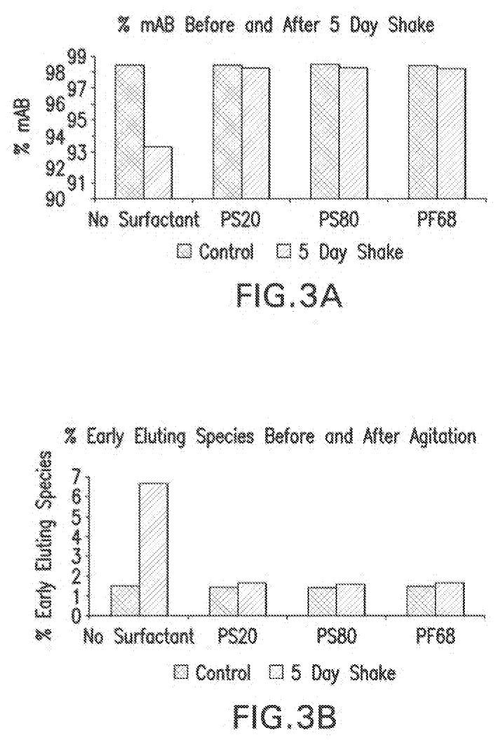

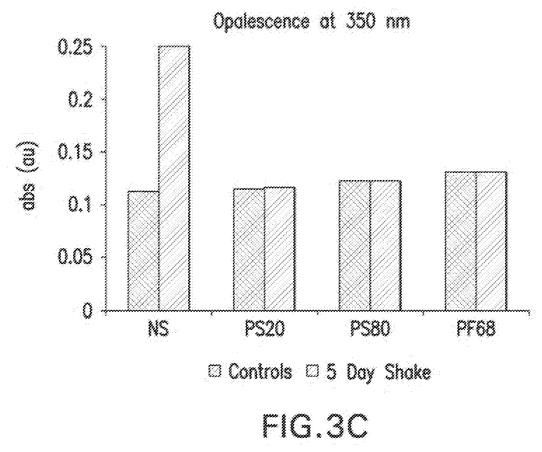

Various excipients (100 mM NaCl, 7% sucrose, 7% trehalose and 6% mannitol) were also tested for their effects on unfolding temperature (FIG. 2A) and opalescence (FIG. 2B). Seven percent sucrose was added to render formulations isotonic, to decrease opalescence, and to increase thermal stability (increase Tm). The surfactants polysorbate-20 (PS20), polysorbate-80 (PS80) and PLURONIC® F-68 were also tested for their effects on aggregation (FIGS. 3A and 3B) and opalescence (FIG. 3C). Polysorbate 80 was added to minimize aggregation due to agitation-induced s tress.

These results led to a change in preferred buffer system. Anti IL-23 had exhibited self-association and opalescence at high concentrations in citrate pH 5.5 and pH 6.0. Replacing citrate with acetate (pH 5.5) or histidine (pH 6.0) minimized opalescence without compromising thermal and biochemical stability. Sucrose (7%) was added to render the formulations isotonic. Sucrose (7%) also decreased opalescence and increased thermal stability (increased Tm) and decreased percentage loss in monomer during accelerated stability studies (data not shown). Polysorbate 80 (0.05%) was added to minimize aggregation due to shaking stress.

A histidine based formulation comprising 10 mM histidine (pH 6.0), 7% sucrose and 0.05% PS-80 was selected as the preferred high concentration formulation of hum13B8-b. Various solution properties, including viscosity, density, osmolarity, and particulates, were determined for 10 mM histidine (pH 6.0) formulation. See Table 1. The observed room temperature viscosity (5.65 cP) is acceptable for use in prefilled syringes and autoinjectors.

TABLE 1
Solution Properties of Histidine Formulation

| Solution Property | Histidine (pH 6.0) | |
|---|---|---|
| Viscosity (cP) | 5.65 | (25° C.) |
| | 14.46 | (5° C.) |
| Density (g/cm3) | 1.06 | |
| Osmolality | 266 | |
| Sub-visible Particles | 25 | (>10 μm) |
| | 2 | (>25 μm) |

### V. Stability of High Concentration Solution Formulations of Humanized Anti-IL-23p19 Antibody 13B8-b

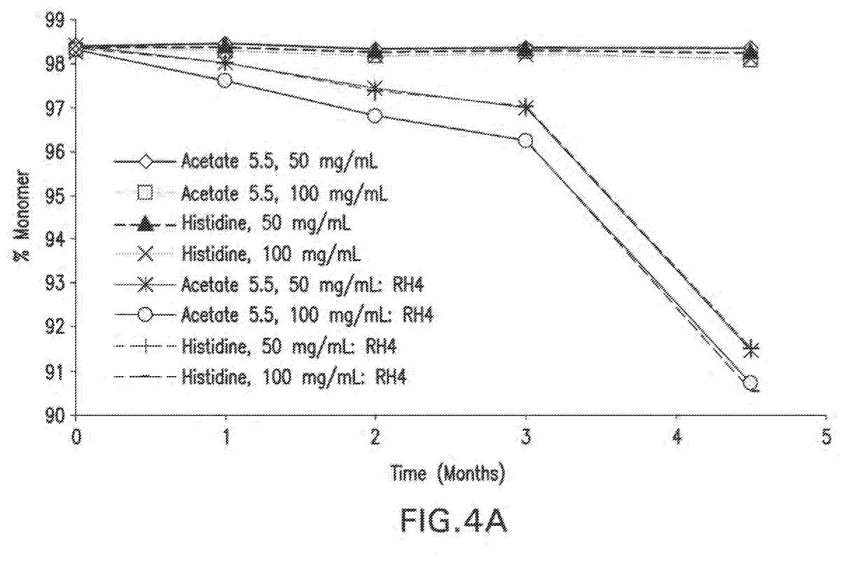

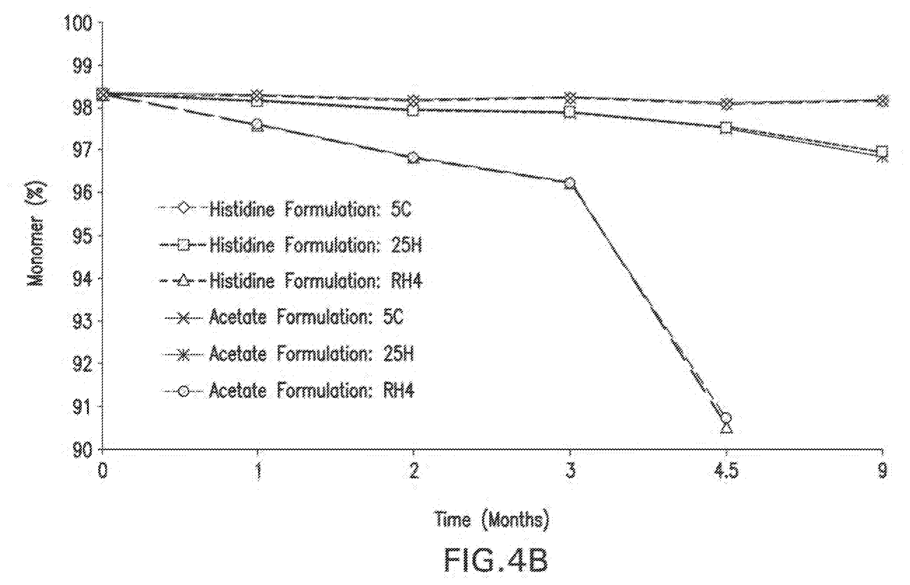

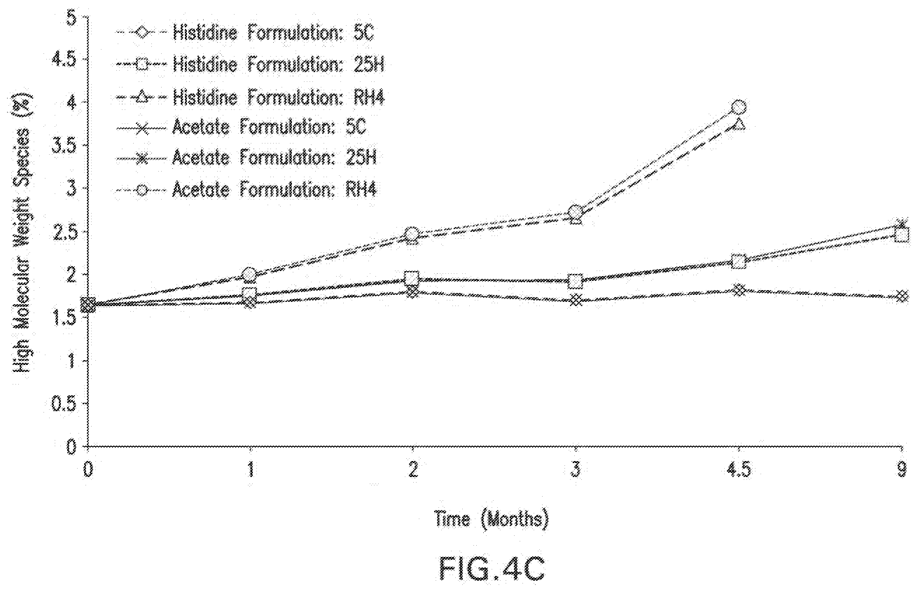

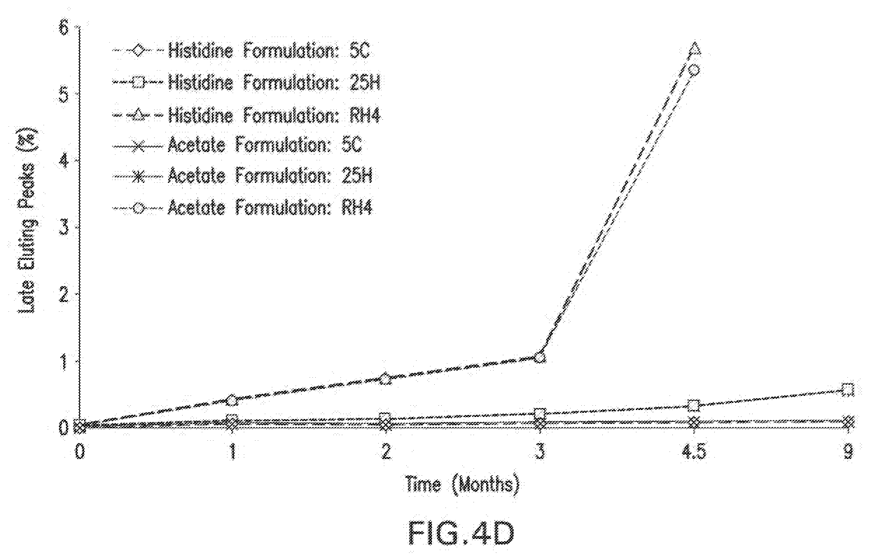

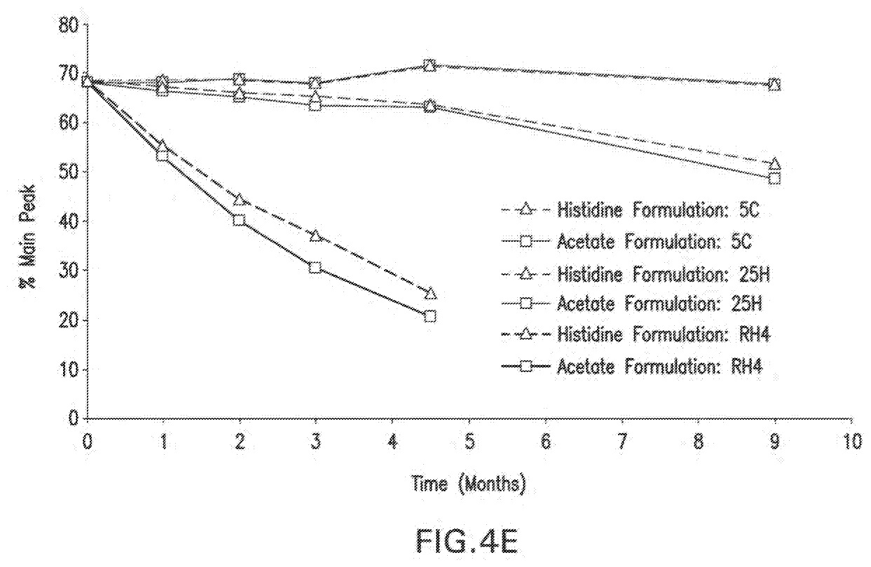

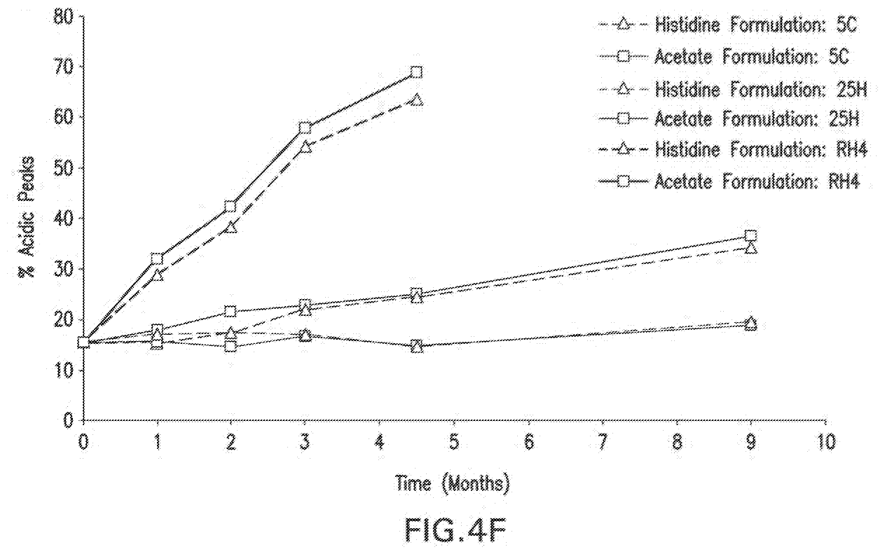

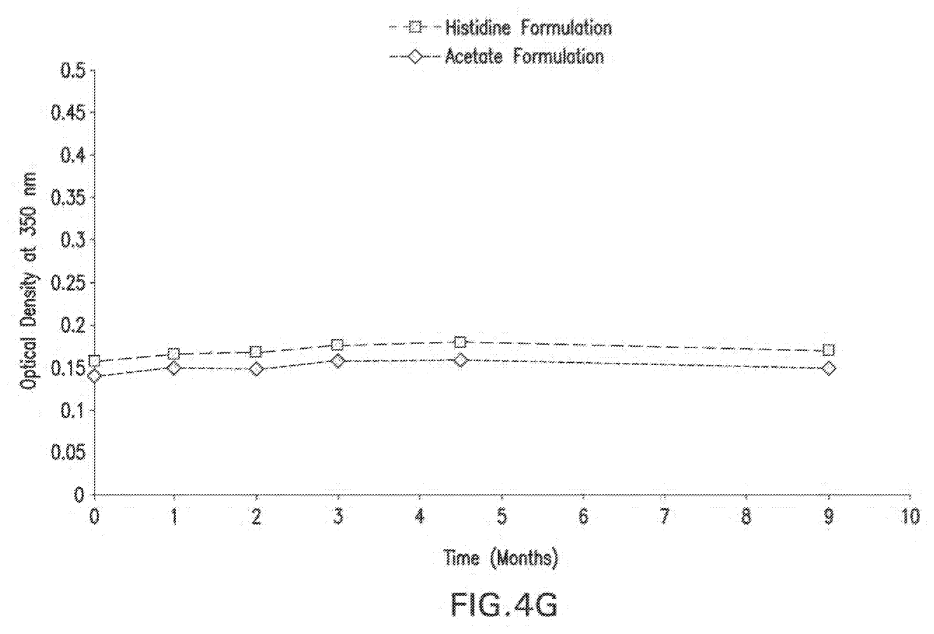

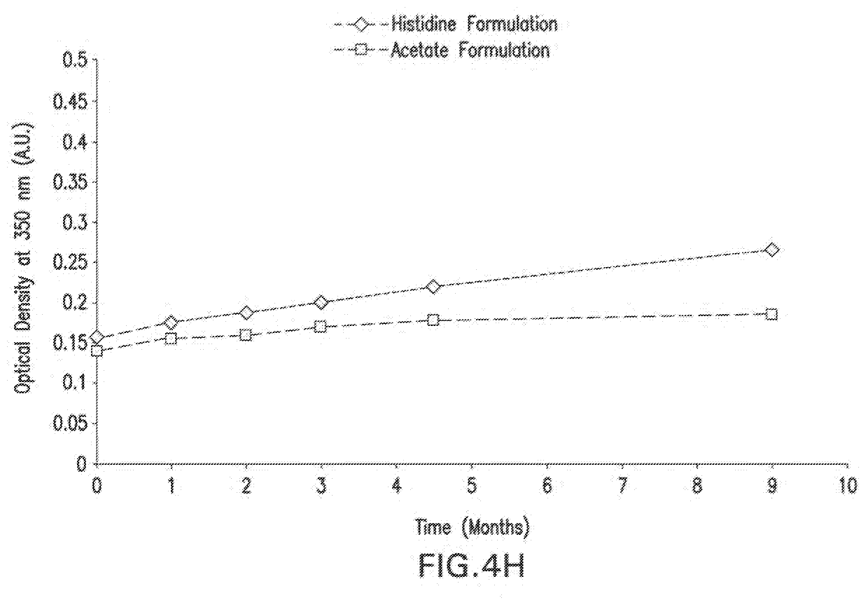

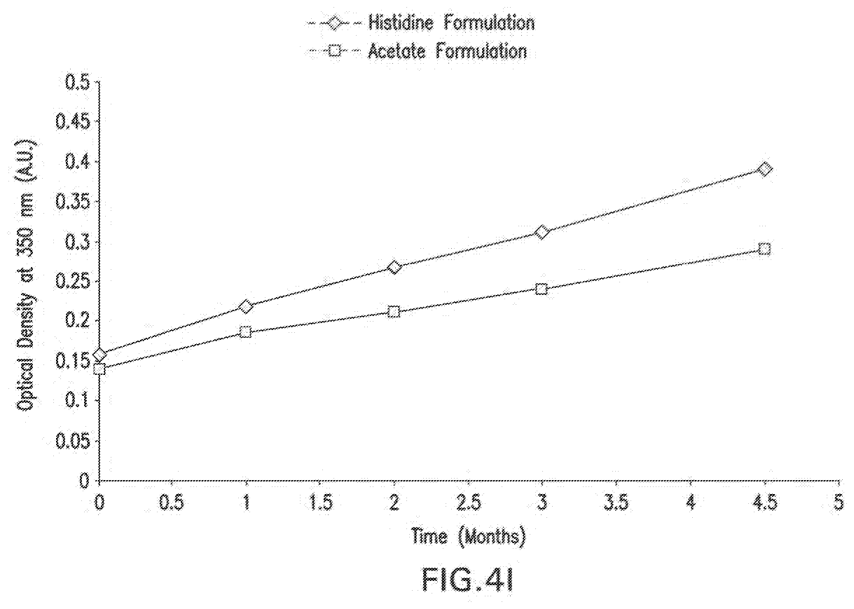

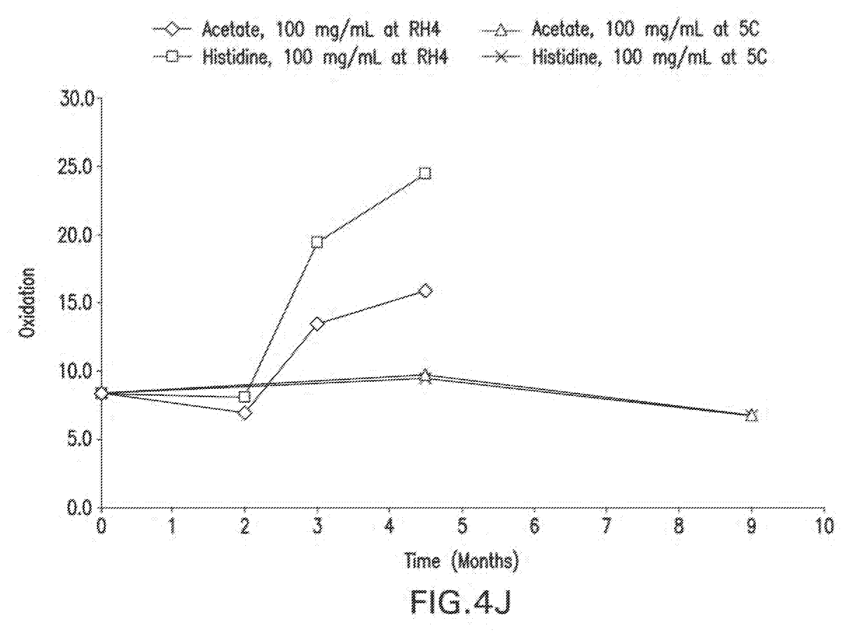

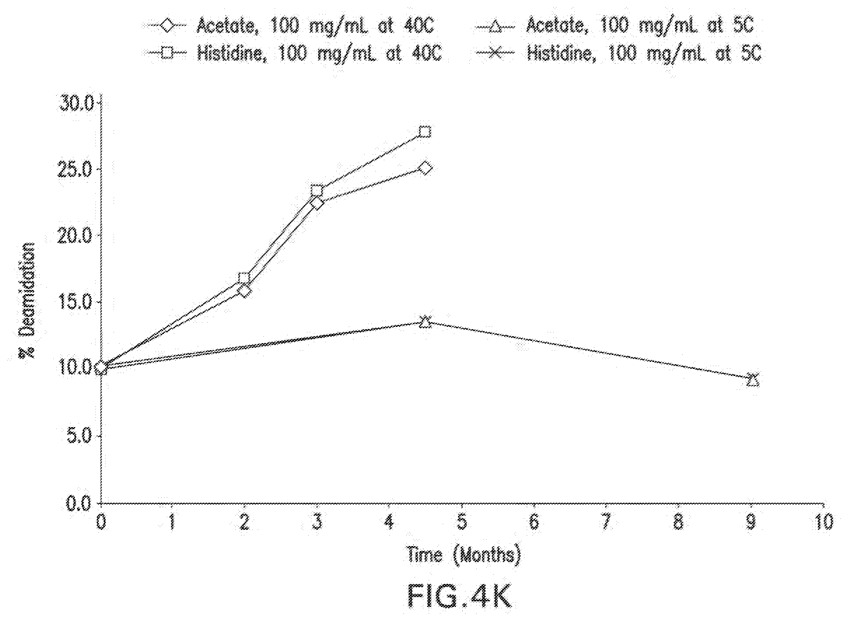

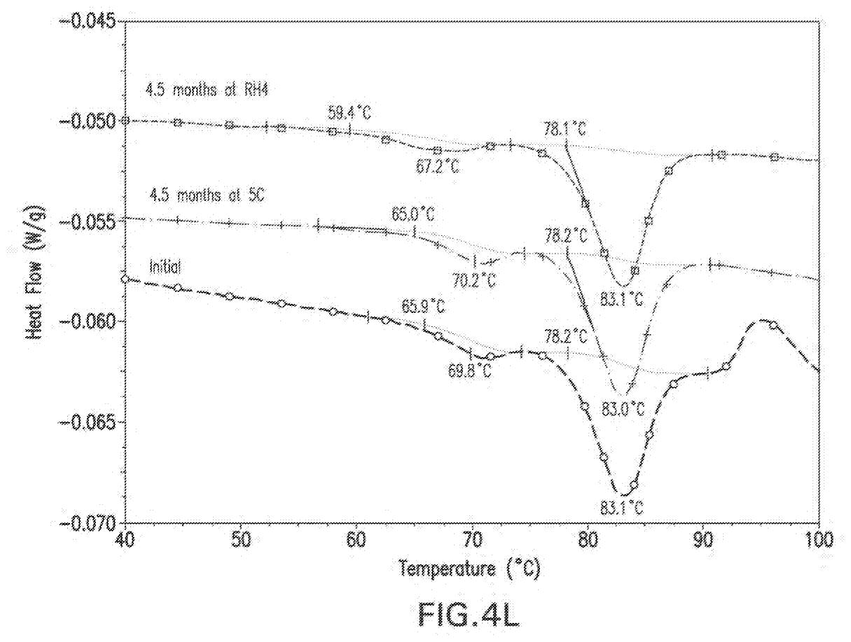

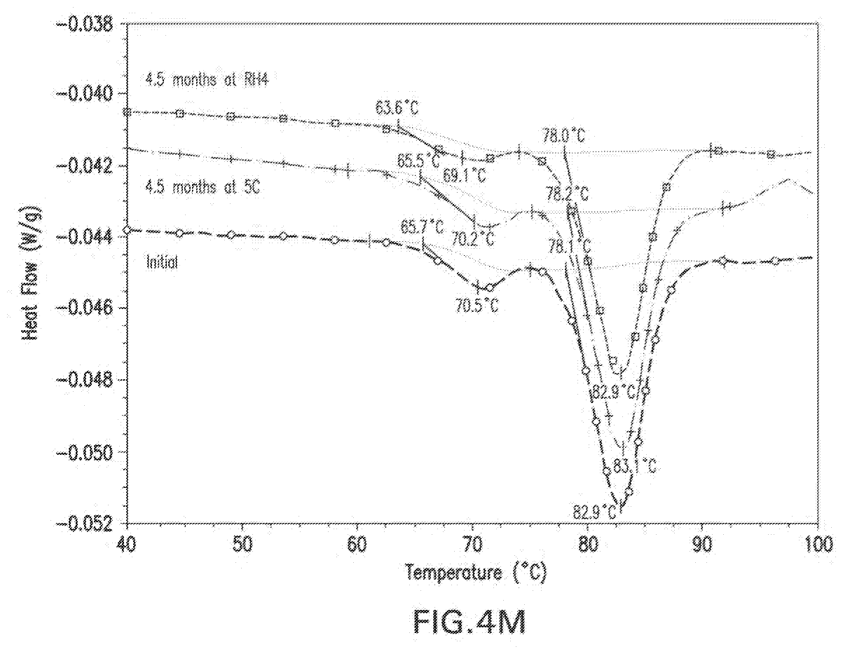

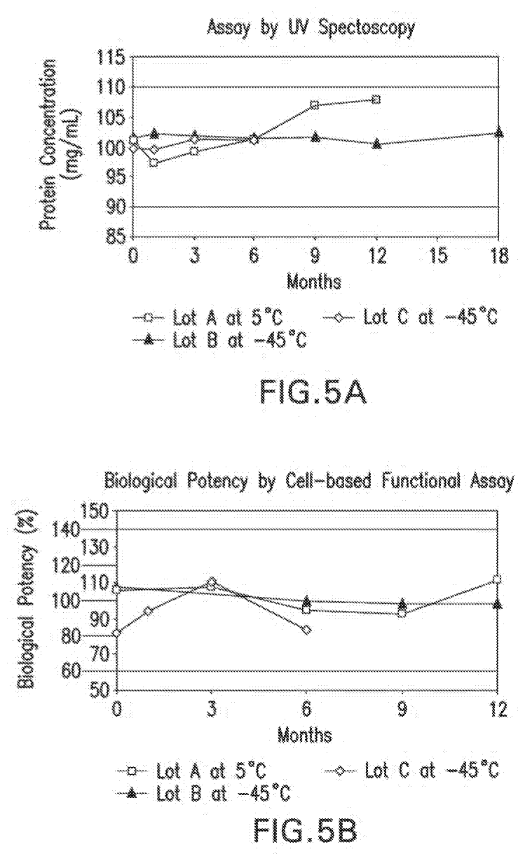

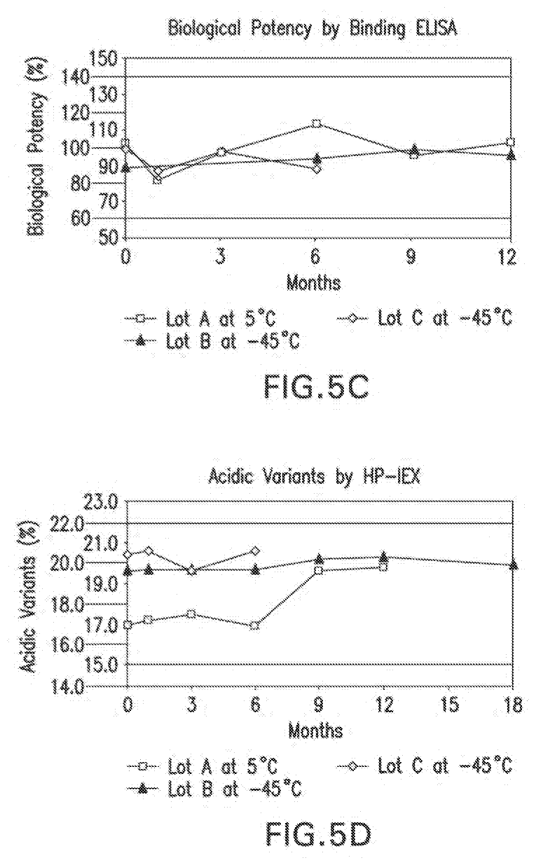

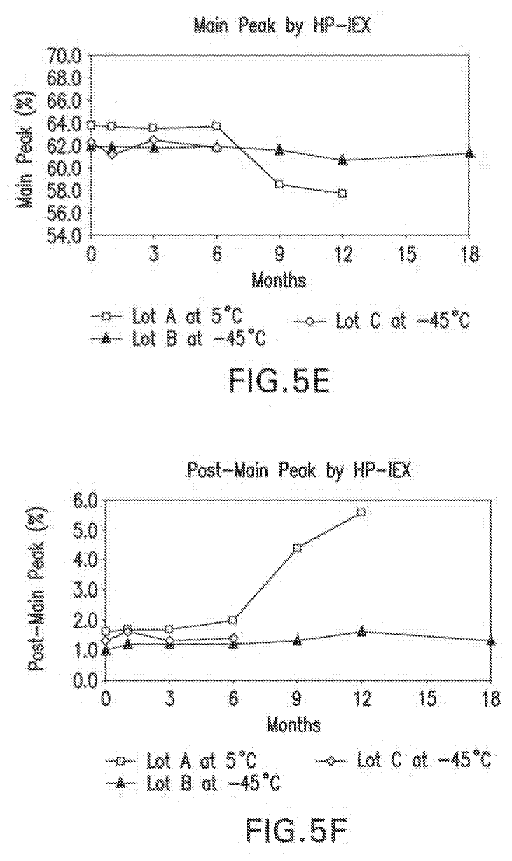

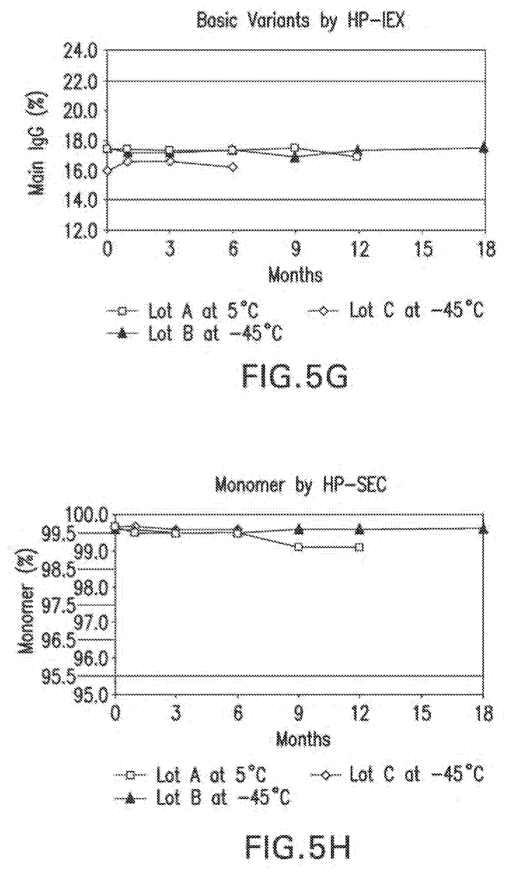

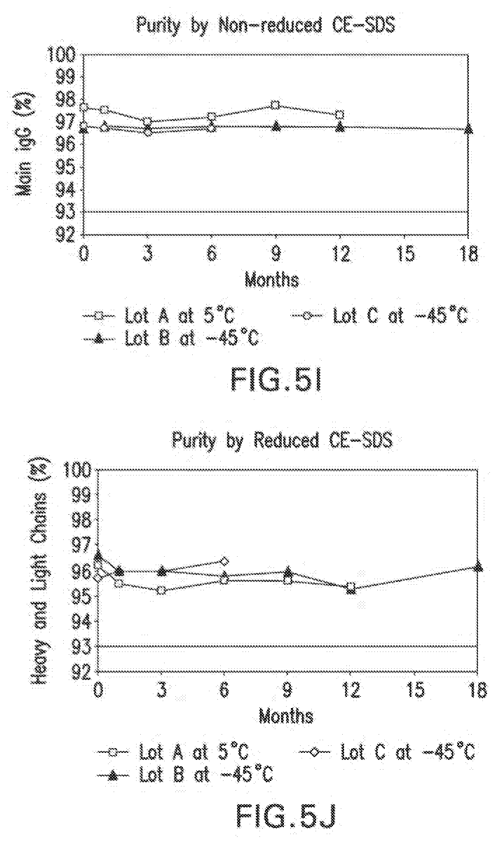

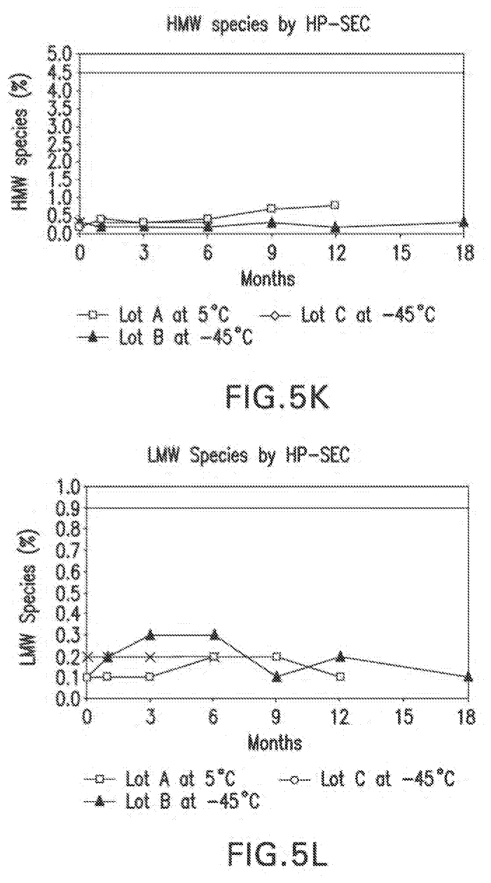

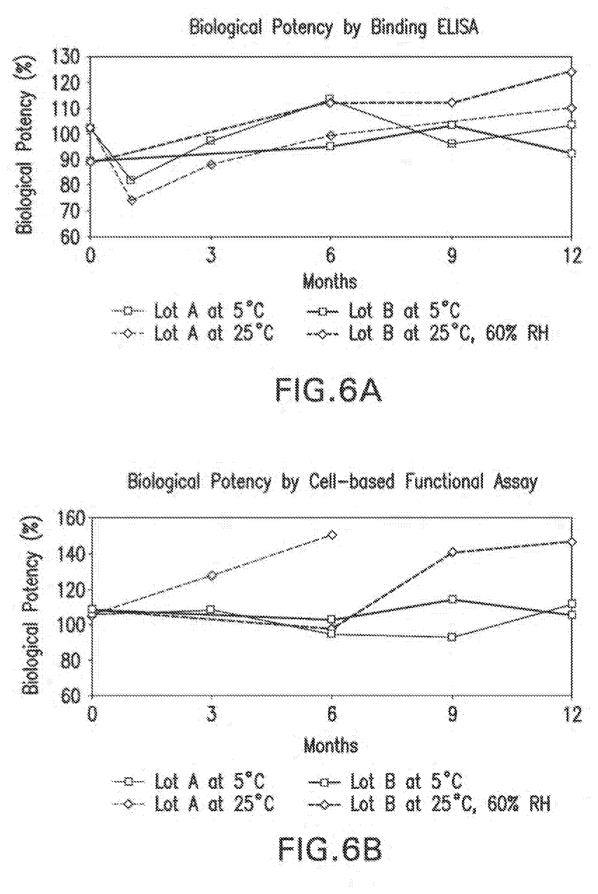

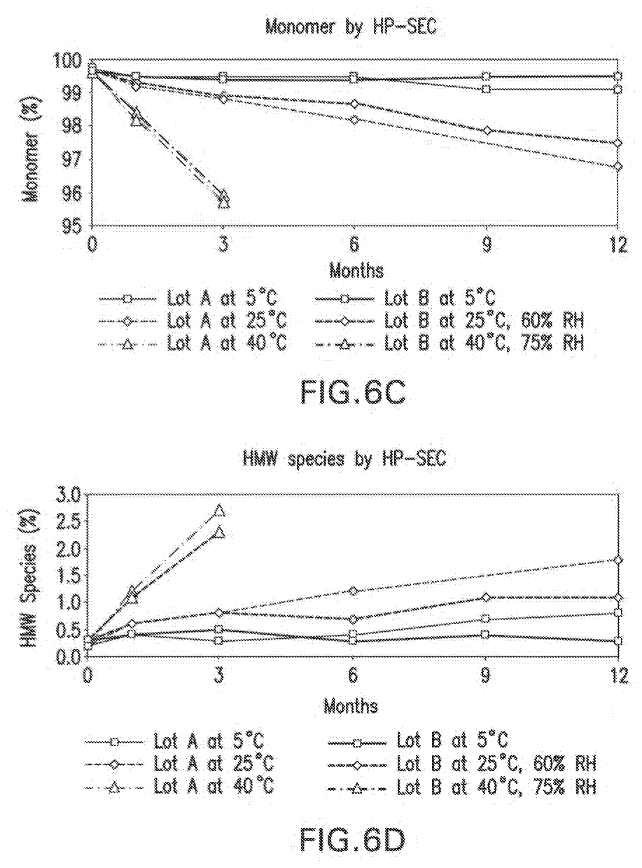

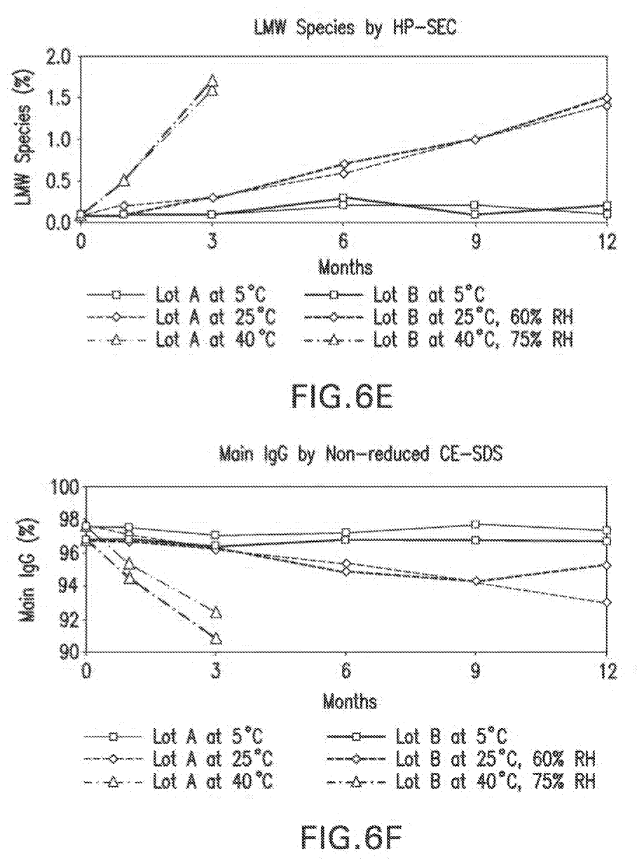

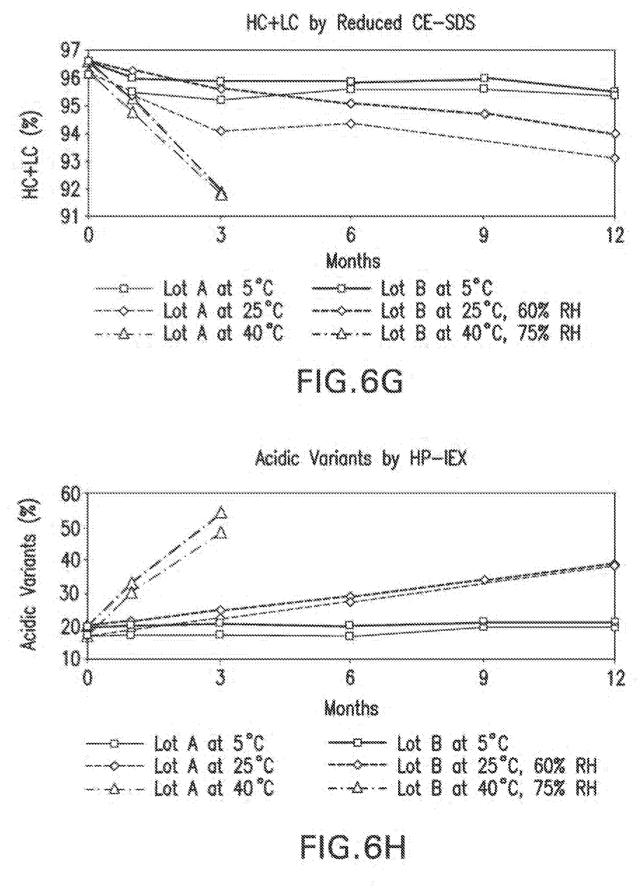

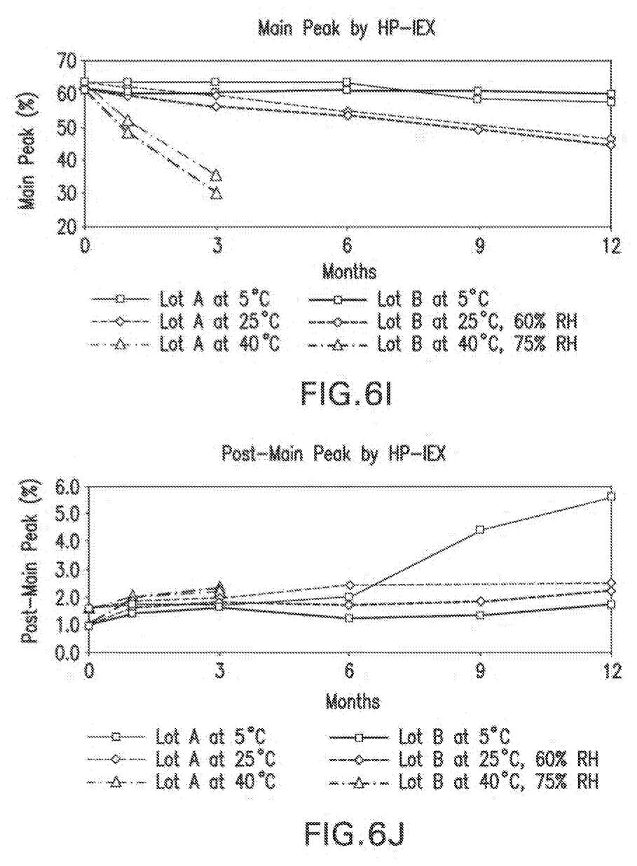

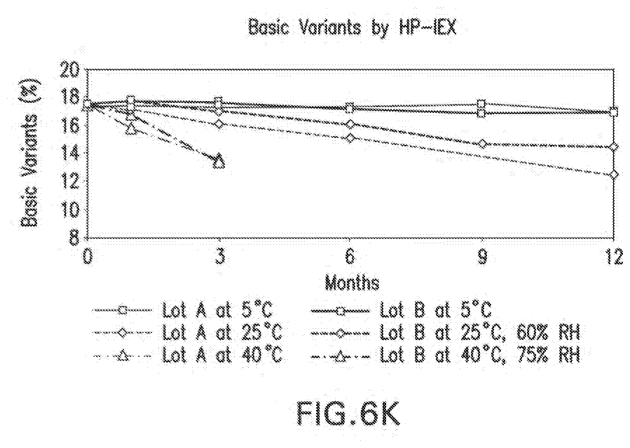

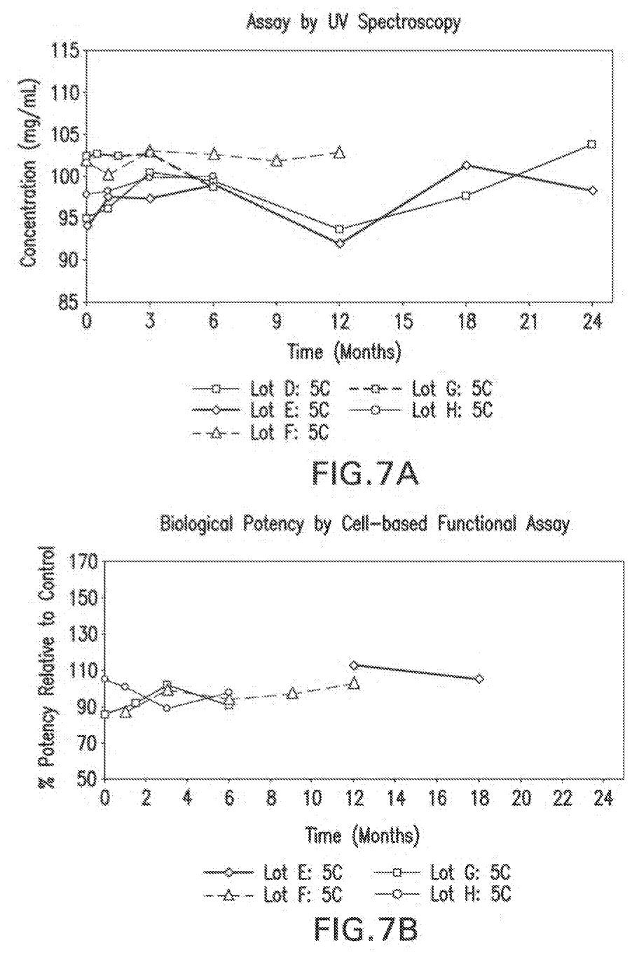

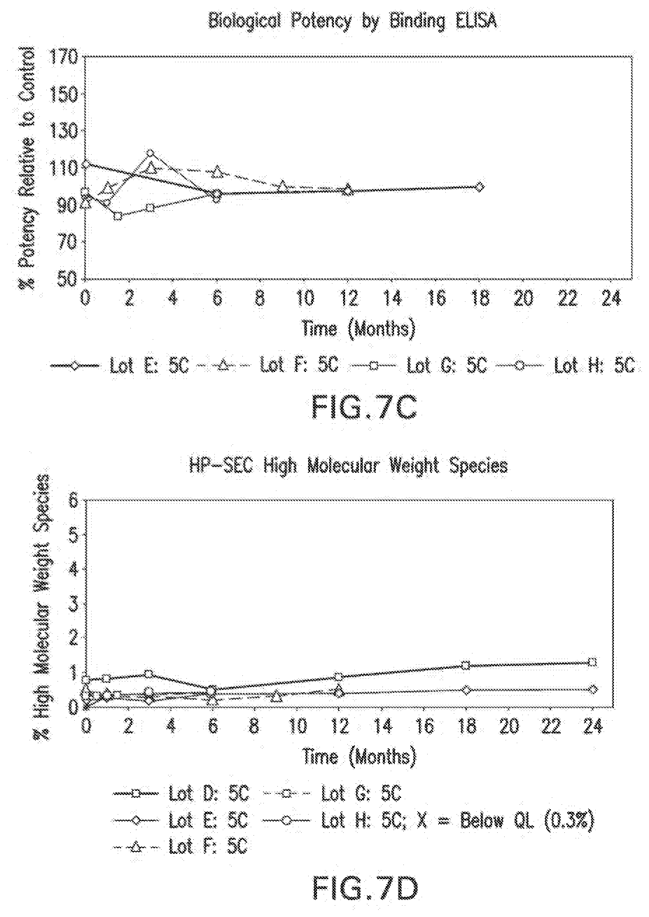

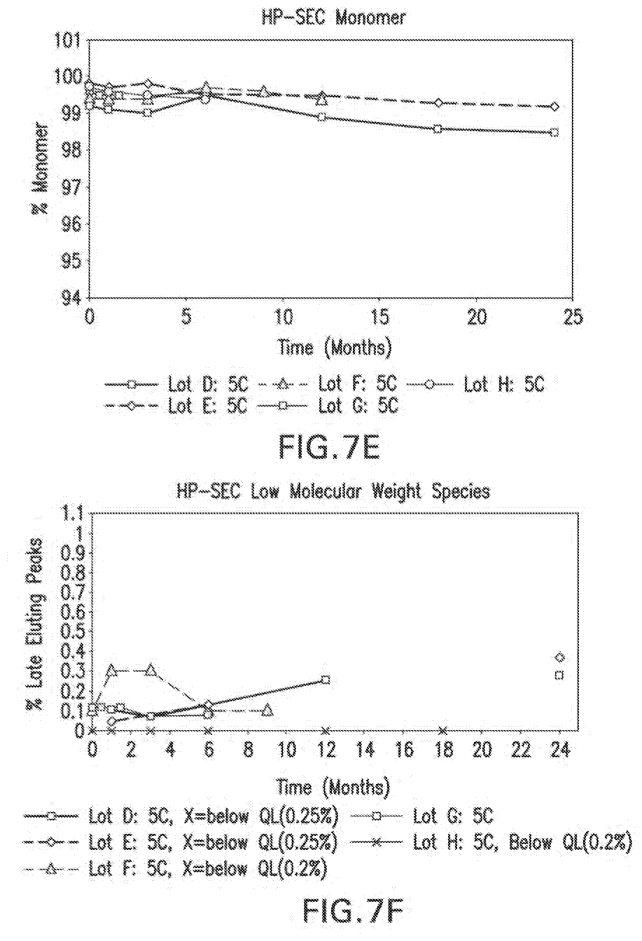

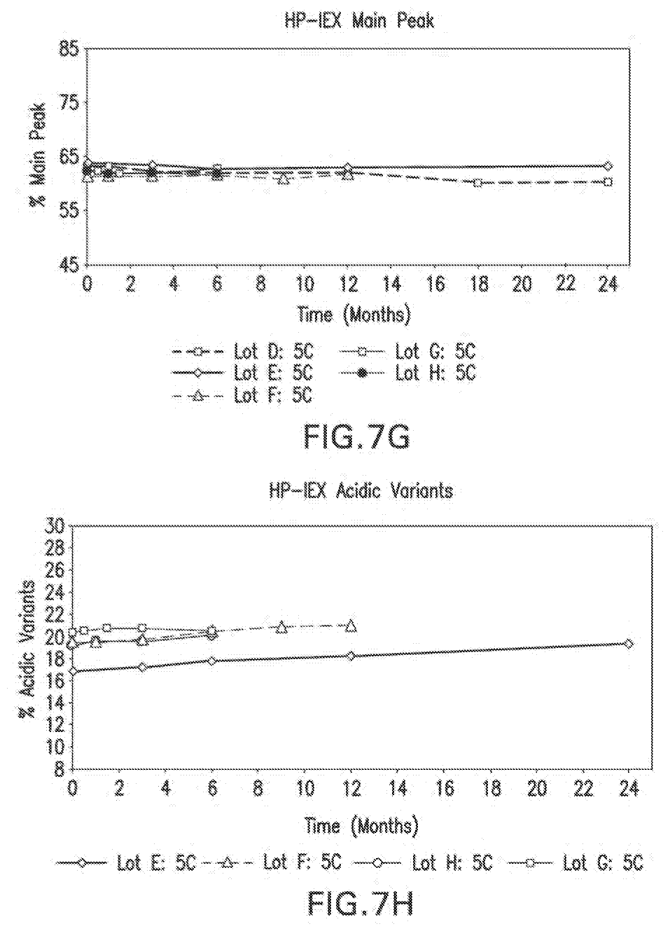

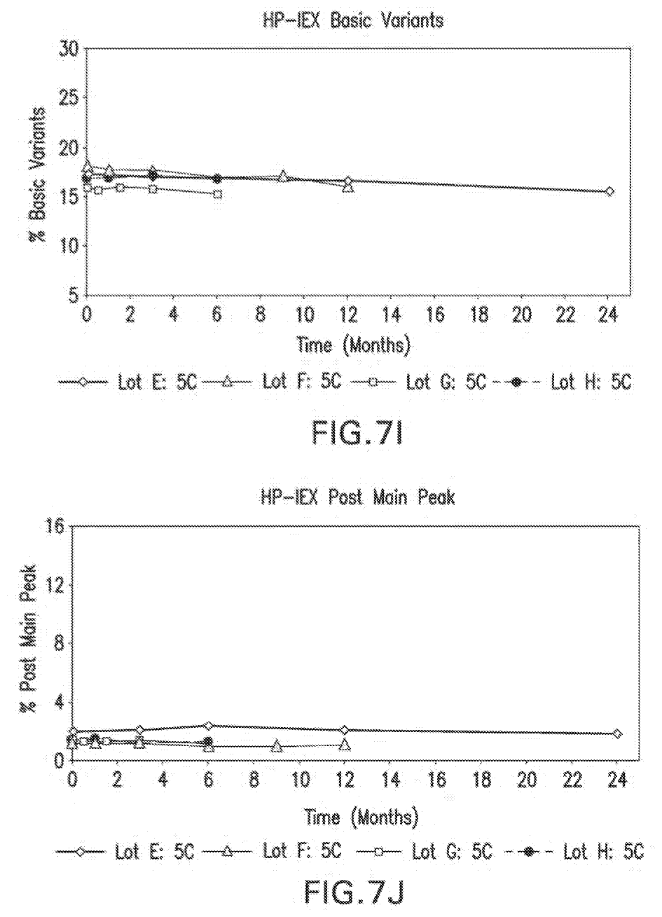

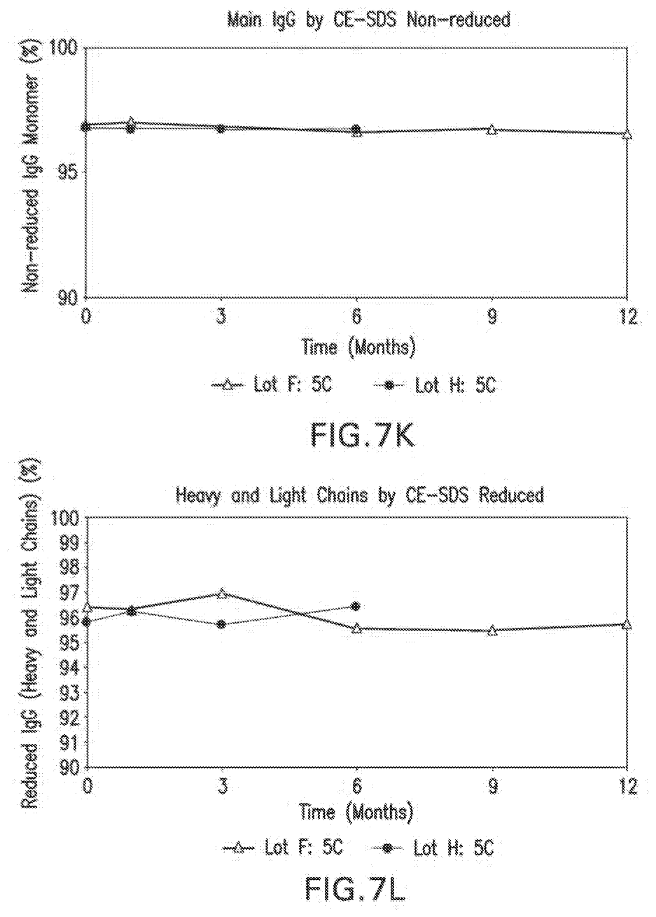

Long term stability of the selected formulations of the present invention was studied after 3-24 months of storage under a variety of storage conditions. Samples were incubated in 2 ml glass vials (FIG. 4), in 30 ml bags (bulk storage) (FIGS. 5 and 6), or in pre-filled syringes (single-dose commercial packaging) (FIG. 7) at a variety of temperatures and humidity levels. Samples were analyzed for the presence of degradation and aggregation products by such methods as high-performance size exclusion chromatography (HP-SEC), ion exchange chromatography (HP-IEX), SDS capillary electrophoresis (CE-SDS, reducing and non-reducing), and peptide mapping. Antibody stability was measured by differential scanning calorimetry (DSC). Biological activity was assessed by IL-23 binding ELISA and a cell-based functional assay. Antibody concentration was determined by UV absorption at 280 nm. Opalescence was determined by measuring optical density at 350 nm (OD₃₅₀). Results are provided at FIGS. 4-7.

In a first set of experiments, 1.5 ml samples were placed in 2.0 ml glass vials and analyzed after storage under various conditions. Results are presented at FIG. 4. Very little degradation or aggregation was observed in samples stored at 5° C. and ambient humidity, which corresponds to typical refrigeration conditions. FIGS. 4A-4F. Accelerated degradation conditions, such as RH4 (40° C., 75% relative humidity), which were included as a positive control for sample degradation, showed the expected loss of monomer and rise of degradation and aggregation products starting as soon at the first time point (one month). FIGS. 4A-4F. Opalescence was stable at 5° C. (FIG. 4G), but increased at 25° C. and 40° C.(FIGS. 4H and 4I). Oxidation was similarly minimal at 5° C., but significant under RH4 conditions, or at 40° C. FIGS. 4J and 4K. These results indicate that although the experiments were capable of detecting degradation by a number of assays, as evidenced, e.g., by the RH4 results, storage under typical refrigeration conditions led to little or no loss in product quality over at least about nine months of storage.

In a second set of experiments, samples were placed in 30 ml ethylene-vinyl-acetate (EVA) fluid contact layer CELSIUS®-Pak bags (Sartorius, Goettingen, Germany) and analyzed after storage under various conditions. Results are presented at FIGS. 5 and 6. These experiments were designed primarily to assess stability of drug substance under typical bulk storage conditions.

Bulk storage samples were stored under three frozen conditions (−80°, −45° C. and −20° C.) and refrigerated (2°-8° C.). Representative data are provided at FIG. 5 for samples stored at 5° C. (±3° C) and −45° C. for up to 18 months. Slight increases in concentration were observed over 12 months of storage (FIG. 5A), presumably due to evaporation from storage bags in the 5° C. samples. There were no significant trends regarding biological activity (FIGS. 5B and 5C), and protein-related impurities, degradation products and aggregates were generally within specifications for up to 18 months, particularly for samples stored frozen at −45° C. (FIGS. 5D-5L).

Stability in bulk storage was also assessed at higher temperatures. Representative data are provided at FIG. 6 for samples stored at 5° C. (±3° C.), 25° C., 25 H, 40° C. and RH4 for up to 12 months. Refrigerated samples showed stable biological activity over 12 months, whereas samples stored at room temperature (25°) actually showed apparent increases in biological potency, an effect that is likely due to net concentration due to evaporation. FIGS. 6A and 6B. Samples were stable over 12 months when stored at 5° C., but protein-related impurities, (degradation products and aggregates) increased over time for samples stored at 25° C. and 25 H, and increased dramatically for samples stored at 40° C. and RH4 (accelerated degradation conditions). FIGS. 6C-6K.

In a third set of experiments, 1 ml samples of 100 mg/ml antibody formulation were placed in syringes (BD Hypak Physiolis Pre-Filled Syringes) and analyzed after storage under various conditions. Results are presented at FIG. 7 for samples stored at 5° C. (±3° C) for up to 24 months. Antibody concentrations remained essentially unchanged (FIG. 7A), as did biological activity (FIGS. 7B and 7C). Levels of high molecular weight species, percent monomer, low molecular weight species, percent main antibody peak, acidic variants, basic variants, post main peak species, main IgG, and heavy and light chains (FIGS. 7D-7L, respectively) all remained essentially stable over at least 12-24 months.

The long term stability experiments presented in FIGS. 4-7 reveal that the histidine formulation of hum13B8-b of the present invention is stable, with regard to both biological activity and physical integrity, at high antibody concentrations under typical storage conditions, whether in frozen in bulk solution or refrigerated, such as in a prefilled syringe.

Results obtained under accelerated degradation conditions, such as RH4 and 25 H, and other elevated temperature conditions, are intended merely to illustrate the possible breakdown products and pathways for antibody hum13B8-b, and do not reflect the degradation rates for antibody intended for therapeutic use. Major degradation routes for hum13B8-b found during accelerated degradation conditions include loss of purity observed by CE-SDS, increases in HMW and LMW species and decrease in the percentage monomer observed by HP-SEC. In addition, HP-IEX revealed increases in acidic variants and post-main peak species, with decreases in basic variants and the main peak. Extended storage of solution formulations of antibody drugs, such as hum13B8-b, at elevated temperature is very unlikely. Long term storage of hum13B8-b, for example, is likely to be stored in CELSIUS®-Pak bags frozen at −45° C., under which conditions quality attributes are expected to remain stable for at least 18 months. Pre-filled syringes or autoinjectors containing individual doses of hum13B8-b are likely to be stored at about 5° C. (±3° C.), and are also expected to remain stable for at least 18 months.

### VI. Dosing and Administration

Although the high concentration solution formulations of the present invention are particularly suitable for high-dose subcutaneous administration, such formulations may also be administered in other ways. Suitable routes of administration may, for example, include oral, rectal, transmucosal, or intestinal administration; parenteral delivery, including intramuscular, intradermal, intramedullary injections, as well as intrathecal, direct intraventricular, intravenous, intraperitoneal, intranasal, or intraocular injections.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Subcutaneous administration may be performed by injection using a syringe, an autoinjector, an injector pen or a needleless injection device.

Although the high concentration solution formulations of the present invention are particularly advantageous for uses requiring a high concentration of antibody, there is no reason that the formulations can't be used at lower concentrations in circumstances where high concentrations are not required or desirable. Lower concentrations of antibody may be useful for low dose subcutaneous administration, or in other modes of administration (such as intravenous administration) where the volume that can be delivered is substantially more than 1 ml. Such lower concentrations can include 60, 50, 40, 30, 25, 20, 15, 10, 5, 2, 1 mg/ml or less.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343: 1594-1602; Physicians' Desk Reference 2003 (Physicians' Desk Reference, 57th Ed); Medical Economics Company; ISBN: 1563634457; 57th edition (November 2002).

Determination of the appropriate dose is made by the clinician, e.g., using parameters or factors known or suspected in the art to affect treatment or predicted to affect treatment. The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. In some circumstances, a low initial does is selected and the dosage is increased by small increments thereafter until the desired or optimum therapeutic benefit is achieved relative to any negative side effects. Important diagnostic measures include those of symptoms of, e.g., the inflammation or level of inflammatory cytokines produced. The protein is suitably administered to the patient at one time or repeatedly. The protein may be administered alone or in conjunction with other drugs or therapies.

Antibodies can be provided by continuous infusion, or by doses at intervals of, e.g., one day, 1-7 times per week, one week, two weeks, monthly, bimonthly, quarterly, semiannually or annually, etc. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

### VII. Uses

The present invention provides high concentration solution formulations of anti-human IL-23p19 mAb hum13B8-b for use in the treatment of inflammatory disorders and conditions, e.g., of the central nervous system, peripheral nervous system, and gastrointestinal tract, as well as autoimmune and proliferative disorders.

The formulations of the present invention can be used in the treatment of, e.g., multiple sclerosis (MS), including relapsing-remitting MS and primary progressive MS, Alzheimer's disease, amyotrophic lateral sclerosis (a.k.a. ALS; Lou Gehrig's disease), ischemic brain injury, prion diseases, and HIV-associated dementia, as well as neuropathic pain, posttraumatic neuropathies, Guillain-Barre syndrome (GBS), peripheral polyneuropathy, and nerve regeneration.

The formulations of the present invention can also be used in the treatment of inflammatory bowel disorders, e.g., Crohn's disease, ulcerative colitis, celiac disease, and irritable bowel syndrome. They can also be used in the treatment of inflammatory disorders such as graft-versus-host disease, psoriasis, atopic dermatitis, arthritis, including rheumatoid arthritis, osteoarthritis, and psoriatic arthritis, autoimmune disorders, such as systemic lupus erythematosus and type I diabetes, and proliferative disorders such as cancer. See, e.g., PCT patent application publications WO 04/081190; WO 04/071517; WO 00/53631; and WO 01/18051.

### EXAMPLES

### Example 1

### Solution Formulation

In one embodiment, the solution formulation of hum13B8-b of the present invention is provided as a 100 mg/mL solution of antibody in 1.0 mL volume in a prefilled syringe, or an autoinjector. The 1.0 mL volume is the extractable volume, rather than the fill volume, which may include sufficient overfill to ensure deliver of the full 1.0 mL dose. An exemplary recipe for 1.0 ml of the formulation of the present invention is provided at Table 2. In one embodiment, a drug product batch is prepared at 15-30 L scale, or about 15,000-30,000 doses.

TABLE 2
1.0 mL Solution Formulation

| Component | Grade | Quantity |
|---|---|---|
| hum13B8-b | product specifications | 100 mg |
| L-Histidine | Ph. Eur./USP | 0.683 mg |
| L-Histidine HCl | Ph. Eur. | 1.17 mg |
| Polysorbate 80 | Ph. Eur./NF/JP | 0.500 mg |
| Sucrose | Ph. Eur./NF/JP | 70.0 mg |
| Water for Injection | USP | q.s. |

In some embodiments the amounts of the buffer components (L-histidine and L-histidine HCl) deviate slightly from the weight amounts listed in Table 2 due to the need to adjust the pH to approximately 6.0. Specifically, in some embodiments more or less of either the acidic or basic forms of histidine, as compared with the amounts in Table 2, are added to adjust the pH to the desired value of about 6.0. In yet further embodiments amounts of L-histidine and L-histidine HCl approximating those in Table 2 are added to get the pH close to, but a little bit higher than, 6.0, and HCl is then added to lower the pH of the resulting formulation to about 6.0.

### Example 2

### Differential Scanning Calorimetry (DSC)

Valerian-Plotnikov differential scanning Calorimetry (DSC) was used to monitor the thermal stability of antibodies in the formulations of the present invention. DSC directly measures heat changes that occur in proteins during controlled increase or decrease in temperature. A protein in solution is in equilibrium between the native (folded) conformation and its denatured (unfolded) state. DSC is used to determine the temperature at which 50% of the protein is denatured (thermal transition midpoint, Tₘ). Proteins with a higher Tₘ are, in general, more stable. For example, a DSC curve is provided at FIG. 1E, which shows two transitions, the first (Tₘ1) occurring around 64-70° C., depending on the pH, and the second, main transition (Tₘ2) around 80° C. The main transition can be ascribed to unfolding of the Fc fragment. The thermal events preceding the main transition (i.e. at lower temperature) are likely due to unfolding of several other domains within the antibody (e.g. Fab), which is in line with the expected structural elements of IgG1 antibodies described in literature. Vermeer (2000) Biophys. J. 78:394.

### Example 3

### High Performance Size Exclusion Chromatography (HP-SEC)

The percentage of high molecular weight (HMW) species, low molecular weight (LMW) species and monomer in hum13B8-b formulations of the present invention were detected by HP-SEC. The test solution was diluted and separated by HPLC using a size exclusion column (YMC-pack Diol-200, 200 Å pore size, 300×8.0 mm, 5 μm, or equivalent; YMC Co. Ltd., Kyoto, Japan). The peak areas are used to determine the percentage monomer, HMW species and LMW species.

Elution peaks from SEC were characterized using multi-angle laser light scattering (SEC-MALLS), which can be used to estimate the molecular weight and to monitor aggregates. After separation of the monomer peak from fragments and aggregates on the SEC column, the sample is passed through ultra violet (UV), MALLS and refractive index (RI) detectors, enabling the calculation of analyte concentration and subsequent estimation of its molecular weight (MW). The intensity of the scattered light (detected by MALLS) is proportional to the product of the protein concentration (determined by RI) and the molecular weight. SEC-MALLS shows a predominant main peak with a molecular weight of approximately 138 kDa. This corresponds fairly well with the calculated theoretical molecular weight of the monomer of hum13B8-b, and also the mass of the monomer of hum13B8-b as detected using mass spectrometry, when taking the chromatographic resolution of SEC and the accuracy of light scattering detection into account. A high molecular weight species (HMW1), which has an molecular weight of approximately 300 kDa, likely represents a dimer species. A second HMW peak (HMW2), which is not observed in all batches, contains species with an estimated molecular weight of approximately 465 kDa, corresponding to a trimer species. A low molecular weight species (LMW) is detected with an approximate molecular weight of 108-117 kDa, which may represent a fragmentation product, for example, the product of hinge fragmentation.

### Example 4

### Dynamic Light Scattering (DLS)

Dynamic light scattering (DLS) is a technique that can provide insight into the size distribution profile of monoclonal antibodies in a range of 0.5 nm to 6 μm in solution. Monoclonal antibody hum13B8-b shows an average hydrodynamic diameter of approximately 9.5 nm in intensity (and 6.5 nm in volume). This is consistent with values for other monoclonal antibodies. Higher ionic strength formulations tend to have lower hydrodynamic diameters than low ionic strength formulations, and can be influenced by other solution parameters such as buffer and pH. See FIGS. 1I and 1J. Antibodies in the formulations of the present invention are essentially monodisperse. The hydrodynamic diameter for the antibodies of the present invention is reversible, as evidenced by experiments in which an antibody is diluted into a buffer of higher or lower ionic strength (data not shown). These results suggest that the hydrodynamic diameter will equilibrate to the local surroundings of the injection site when administered to a subject.

### Example 5

### High Performance Cation Exchange Chromatography (HP-IEX)

Charge variants for hum13B8-b were detected by HP-IEX, which relies on electrostatic interactions between proteins in a sample and charges immobilized on a resin. Positively charged hum13B8-b variants are bound to a negatively charged resin on a weak cation exchange column (Dionex ProPac WCX-10, 4×250 mm, or equivalent; Thermo Scientific, Bannockburn, Ill., USA) and are separated by HPLC. Antibody is eluted by increasing the pH and the salt concentration, effectively decreasing the charge of the antibody variants and replacing them with ions of equivalent charge. The presence of hum13B8-b and variants in the eluent are determined by UV detection. The peak areas are used to determine the percentage Acidic Variants, Main Peak, Post-Main Peak and Basic Variants.

All species detected before the main peak, eluting around 23 minutes, are referred to as Acidic Variants, while those eluting after the Main Peak are referred to as Basic Variants. Analysis of the Acidic Variants and Acidic Peaks reveals that they have no N- or C-terminal modifications, whereas the Main Peak exhibits full lysine cleavage at the C-terminus and full pyroglutamate formation at the N-terminus. The Main Peak retains full biological activity. The Post-Main Peak exhibits full lysine cleavage and limited proline α-amidation at the C-terminus, full pyroglutamate formation at the N-terminus, oxidation of Met251 and Met427, and exhibits reduced potency as measured by binding ELISA. Basic Peak 1 exhibits full lysine cleavage and some proline α-amidation at the C-terminus, and full pyroglutamate formation at the N-terminus. Basic Peak 3, the most abundant of the Basic Peaks, exhibits full lysine cleavage at the C-terminus and incomplete pyroglutamate formation at the N-terminus. Basic Variants exhibit full lysine cleavage and variable levels of proline α-amidation at the C-terminus, and incomplete to complete pyroglutamate formation at the N-terminus. The majority of charge variants retain biological activity.

### Example 6

### Peptide Mapping

Peptide mapping provides information on the primary structure of hum13B8-b, and can be used to assess whether oxidation has taken place. To increase sequence coverage two different enzymatic peptide maps were used to characterize hum13B8-b. The first was peptide mapping after endoproteinase Lys-C digestion. A peptide map was obtained by treating a sample of hum13B8-b with guanidine hydrochloride to denature the protein, and with 1,4-dithiothreitol (DTT) to reduce disulfide bonds. Samples were then treated with iodoacetamide (IAM) to alkylate free thiols resulting from the DTT treatment. Samples were then digested with endoproteinase Lys-C. Analysis of the resulting peptides was performed using reversed-phase liquid chromatography (Xbridge C18, 5 μm, 130 Å pore size, 2.1×150 mm or equivalent; Waters Corporation, Milford, Mass., USA) coupled to electrospray mass spectrometry. The peptide sequences originating from the light (L) and heavy (H) chains, as well as their masses that were detected using mass spectrometry, are given in Table 3. Retention times were determined by ultraviolet absorption at 214 nm during chromatography over 90 minutes. Masses were corrected for the alkylation of cysteine residues by adding a net mass of 57 Da per IAM-alkylated cysteine residue. Five peptides originating from the complementarity-determining region (CDR) of hum13B8-b are of particular interest. Common post-translationally modified peptides, such as the pyroglutamated N-terminal heavy chain peptide, the lysine-clipped C-terminal heavy chain peptide and several methionine-oxidized and deamidated peptides were observed as well. None of these modifications are found in the CDRs of the molecule. Routine peptide mapping experiments may be performed without use of mass spectrometry, and retention times of observed peaks can be used to compare samples, such as a stability (test) sample and a control (reference) sample.

A second peptide mapping method, employing trypsin digestion, was developed to add coverage over residues 64-120 of the heavy chain, which was not well represented in the Lys-C peptide map. In addition to the cleavage site C-terminal of lysine residues that is shared with endoproteinase C, trypsin also cleaves C-terminal of arginine residues (unless the next residue is P), resulting in a set of smaller peptides. The peptides arising from residues 64 to 120 of the heavy chain were detected using reversed phase liquid chromatography coupled to UV absorbance detection and mass spectrometry (for peak annotation). Relevant tryptic peptide fragments of hum13B8-b are included in Table 3.

TABLE 3
Peptide Mapping with Endoproteinase Lys-C and Trypsin

| | | Retention | Observed | |
|---|---|---|---|---|
| Chain | Peptide | time (min) | mass (Da) | I. Remarks |
| H | 13-19 | 17.15 | 685.42 | |
| H | 334-337 | 8.94 | 447.27 | |
| H | 213-217 | 13.12 | 599.37 | |
| H | 14-19 | 17.15 | 557.32 | |
| H | 409-413 | 23.95 | 574.34 | |
| H | 439-445 | 35.96 | 659.35 | C-terminus-K |
| H | 326-333 | 37.23 | 837.50 | |
| H | 1-12 | 41.37 | 1267.68 | 1pE |
| H | 340-359 | 43.49 | 2310.19 | |
| H | 338-359 | 43.49 | 2509.32 | |
| H | 133-146 | 43.81 | 1320.68 | |
| | | Retention | Observed | |
| Chain | Peptide | time (min) | mass (Da) | II. Remarks |
| H | 414-438 | 45.04 | 3059.36 | M427 oxidized |
| H | 360-369 | 47.39 | 1160.62 | |
| H | 121-132 | 47.97 | 1185.65 | |
| H | 274-287 | 50.08 | 1676.80 | |
| H | 414-438 | 52.65 | 3043.37 | |
| H | 370-391 | 56.90 | 2544.09 | N383 isoD |
| H | 370-391 | 57.27 | 2543.10 | |
| H | 370-391 | 58.13 | 2544.10 | N383 D |
| H | 248-273 | 60.30 | 2970.41 | M251 oxidized |
| H | 392-408 | 62.47 | 1872.92 | |
| H | 248-273 | 63.11 | 2954.42 | |
| H | 288-316 | 70.98 | 5066.36 | |
| H | 288-316 | 71.19 | 4904.28 | |
| H | 288-316 | 71.39 | 4758.17 | |
| H | 222-247 | 71.74 | 2843.43 | |
| H | 274-325 | 73.05 | 6096.43 | |
| H | 222-245 | 73.88 | 2618.29 | |
| H | 147-209 | 82.40 | 6712.66 | |
| H | 13-63 | 93.78 | 5696.78 | |
| H | 20-63 | 95.45 | 5029.36 | CDR-derived |
| H | 24-63 | 96.01 | 4555.12 | CDR-derived |
| L | 184-188 | 9.76 | 624.28 | |
| L | 208-214 | 19.92 | 868.36 | |
| L | 104-107 | 22.96 | 487.30 | |
| L | 146-149 | 26.46 | 559.32 | CDR-derived |
| L | 150-169 | 29.11 | 2134.95 | |
| L | 191-207 | 39.01 | 1874.92 | |
| L | 46-52 | 40.79 | 833.51 | CDR-derived |
| L | 170-183 | 47.39 | 1501.76 | |
| L | 108-126 | 64.81 | 2101.11 | |
| L | 1-42 | 68.14 | 4765.28 | |
| L | 127-145 | 68.79 | 2125.05 | |
| L | 1-39 | 68.90 | 4483.13 | |
| L | 53-103 | 77.20 | 5559.57 | CDR-derived |

### Example 7

### Protein Concentration by UV Spectroscopy

The protein concentration of antibody hum13B8-b in solution formulations of the present invention is determined by UV spectroscopy. The determination is based on the absorbance of UV light at 280 nm by amino acids like tryptophan, tyrosine and cysteine residues. The absorbance at 280 nm is corrected for light scattering using the absorbance at 320 nm. The method comprises a gravimetrical dilution of the sample in water, and recording of a UV spectrum to establish the absorbance at 280 nm and the absorbance at 320 nm. These absorbance values and the experimentally determined extinction coefficient of 1.44 mL mg⁻¹ cm⁻¹ are used to calculate the hum13B8-b concentration.

### Example 8

### Biological Activity—Cell-Based Functional Assay

A functional cell-based assay was developed to assess the ability of hum13B8-b in formulations of the present invention to block biological activities of human IL-23. This assay evaluates the ability of hum13B8-b to inhibit the IL-23-induced STAT3 activation in an IL-23 responsive cell line (Kit 225). Serial dilutions of a hum13B8-b reference material and test sample are incubated with a fixed concentration of human IL-23, followed by incubation with Kit225 IL-23-responsive cells. Inhibition of STAT3 phosphorylation by hum13B8-b is measured in cell lysates by ELISA using STAT3 capture and anti-p-STAT3 detection antibody pair, followed by incubation with peroxidase conjugated anti-IgG and addition of chemiluminescent substrate. An inhibition response curve (“standard curve”) is generated using a non-linear regression four parameter logistic fit, where the IC50 value represents the concentration of anti-IL-23 that inhibits 50% of the maximum response. Relative potency of the test sample is assessed by comparison of the inhibition response curve of the test sample to the standard curve of and calculated as percent of reference. Relative potency values for multiple replicates of the same sample are combined into a single reportable value—a geometric mean of the relative potency.

### Example 9

### Biological Activity—Binding ELISA

The affinity of hum13B8-b in the formulations of the present invention for human IL-23 is assessed in an equilibrium binding ELISA, where serial dilutions of reference material and test samples are applied to assay plates coated with human IL-23 cytokine. This assay may be used, for example, to assess retention of biological activity in various potential therapeutic formulations. Relative potency of the test sample is assessed by comparison of the dose response curve of the test sample to the dose response curve of reference material and calculated as percent of reference.

ELISAs are performed by methods well known in the art. Briefly, serial dilutions of hum13B8-b in a formulation of the present invention are added to wells of a microtiter plate that had been previously coated with human IL-23 protein and then blocked. After an incubation period, wells are washed and a peroxidase-conjugated goat anti-human IgG (Fc) detection antibody reagent is added. Wells are again washed, and a chemiluminescent peroxidase substrate is added. Signal is read out by chemiluminescence. A sigmoidal dose response curve is generated using non-linear regression four parametric logistic fit, where the EC50 value represents concentration needed to achieve 50% of the maximum IL-23 binding signal. Relative biological potency is calculated by comparing results obtained with test samples with a standard curve based on signal levels obtained with a reference sample. Results are presented as percent biological potency as compared with the reference antibody solution. Relative potency values for multiple replicates of the same sample are combined into a single reportable value—a geometric mean of the relative potency.

### Example 10

### SDS Capillary Electrophoresis (CE-SDS)

Capillary electrophoresis with sodium dodecyl sulfate (CE-SDS) separates proteins based on size. CE-SDS can be performed under non-reducing conditions or under reducing conditions. Non-reducing CE-SDS resolves intact antibodies from other species in the sample, whereas reducing CE-SDS resolves the dissociated heavy and light chains from each other, and also from other potential species in the sample. See, e.g., Rustandi et al. (2008) Electrophoresis 29:3612.

Non-reducing CE-SDS involves heat denaturation of samples of antibody hum13B8-b in the presence of N-ethylmaleimide, to alkylate free cysteine residues, and SDS. Subsequently, the sample is separated in a capillary containing a replaceable SDS polymer matrix, which provides the sieving selectivity for the separation. All peaks in the test sample are integrated and the peak areas are used to determine the percentage Main IgG (intact antibody) in the sample.

Reducing CE-SDS involves heat denaturation of samples of antibody hum13B8-b in the presence of 2-mercaptoethanol, to reduce disulfide bonds, and SDS. Subsequently, the sample is separated in a capillary containing a replaceable SDS polymer matrix, which provides the sieving selectivity for the separation. All peaks in the test sample are integrated and the peak areas are used to determine the percentage intact heavy and light chains in hum13B8-b.

### Example 11

### Sedimentation Velocity Analytical Ultracentrifugation (SV-AUC)

SV-AUC was used to investigate the quaternary structure of the hum13B8-b antibody in the formulations of the present invention. SV-AUC measures the rate at which molecules sediment in response to a centrifugal force. This sedimentation rate provides information on the molecular weight of molecules present in the sample. Hum13B8-b predominantly sediments as one species with a sedimentation coefficient (s20,w) of 7.0S. This species has an estimated molecular weight of approximately 150 kDa, which is in line with the expected molecular weight of the monomer. The frictional ratio, which is dependent on the hydration and shape of the macromolecule, is similar between lots.

### Example 12

### Extended Stability of Antibody Formulations

Additional long-term stability data were obtained for antibody formulations of the present invention. Exemplary stability data up to 24 months are presented in Tables 4-9 below for Lots E and D.

TABLE 4
Storage Condition 5 C.

| Lot D | Stability Test Interval (Months) | | | | | | |
|---|---|---|---|---|---|---|---|
| Test | Initial | 1 M | 3 M | 6 M | 12 M | 18 M | 24 M |
| Assay - UV (A280 nm) mg/mL | 95.1 | 96.1 | 100.4 | 99.4 | 93.6 | 97.7 | 103.8 |
| HP-IEX [%] | | | | | | | |
| Acidic Variants | 10.1 | 10.2 | 10.2 | 10.6 | 9.3 | 11.5 | 11.6 |
| Acidic 1 Peak | 9.7 | 9.8 | 10.7 | 10.5 | 10.7 | 11.4 | 11.5 |
| Pre-Main Peak | | | | | | | |
| Main peak | 63.2 | 63.3 | 62.3 | 62.0 | 62.2 | 60.3 | 60.4 |
| Post-Main Peak | | | | | | | |
| Basic 1 peak | 9.3 | 8.9 | 8.9 | 9.2 | 9.7 | 9.0 | 8.7 |
| Basic 2 peak | 3.9 | 4.0 | 4.4 | 3.9 | 2.9 | 3.4 | 3.1 |
| Basic variants | 3.8 | 3.2 | 3.5 | 3.8 | 5.3 | 3.9 | 4.0 |
| Other | ND | 0.6 | ND | ND | ND | 0.5 | 0.6 |
| HP-SEC [%] | | | | | | | |
| High Molecular Weight Species | 0.79 | 0.83 | 0.95 | 0.51 | 0.84 | 1.16 | 1.24 |
| Monomer | 99.2 | 99.1 | 99.0 | 99.5 | 98.9 | 98.6 | 98.5 |
| Late Eluting Peaks | NQ | 0.11 | 0.07 | NQ | 0.25 | <QL | 0.27 |
| HIAC [particles/mL] | | | | | | | |
| 2 μM | 2827 | 1805 | 11905 | 3701 | 2895 | 1081 | 1879 |
| 5 μM | 292 | 168 | 1474 | 712 | 612 | 201 | 644 |
| 10 μM | 35 | 32 | 326 | 211 | 168 | 36 | 123 |
| 25 μM | 0 | 1 | 13 | 9 | 6 | 2 | 4 |

ND—Not Detected;
NQ—Not Quantified;
QL—Quantification Limit;
5 C.: 5° C. (±3° C.);
QL = 0.25%

TABLE 5
Storage Condition 25H

| Lot D | Stability Test Interval (Months) | | | | |
|---|---|---|---|---|---|
| Test | Initial | 1 M | 3 M | 6 M | 12 M |
| Assay-UV (A280 nm) | 95.1 | 96.4 | 98.6 | 102.8 | 96.3 |
| mg/mL | | | | | |
| HP-IEX [%] | | | | | |
| Acidic Variants | 10.1 | 11.5 | 15.3 | 18.5 | 25.4 |
| Acidic 1 Peak | 9.7 | 11.6 | 13.8 | 14.8 | 17.9 |
| Pre-Main Peak | | | | | |
| Main peak | 63.2 | 60.3 | 56.0 | 51.7 | 42.2 |
| Post-Main Peak | | | | | |
| Basic 1 peak | 9.3 | 9.5 | 8.5 | 8.7 | 8.7 |
| Basic 2 peak | 3.9 | 3.3 | 2.2 | 1.7 | 1.6 |
| Basic variants | 3.8 | 3.8 | 3.6 | 4.5 | 4.2 |
| Other | ND | ND | 0.5 | ND | ND |
| HP-SEC [%] | | | | | |
| High Molecular | 0.79 | 1.47 | 2.05 | 1.35 | 2.69 |
| Weight Species | | | | | |
| Monomer | 99.2 | 98.4 | 97.7 | 97.9 | 96.2 |
| Late Eluting Peak | NQ | 0.14 | 0.30 | 0.77 | 1.15 |

ND-Not Detected;
NQ-Not Quantified;
25H: 25° C./60% Relative Humidity

TABLE 6
Storage Condition RH4

| Lot D | Stability Test Interval (Months) | | | |
|---|---|---|---|---|
| Test | Initial | 1 M | 3 M | 6 M |
| Assay-UV (A280 nm) | 95.1 | 95.0 | 103.6 | 102.7 |
| mg/mL | | | | |
| HP-IEX [%] | | | | |
| Acidic Variants | 10.1 | 20.4 | 46.3 | 58.4 |
| Acidic 1 Peak | 9.7 | 15.9 | 17.8 | 15.0 |
| Pre-Main Peak | | | | |
| Main peak | 63.2 | 49.4 | 24.7 | 15.8 |
| Post-Main Peak | | | | |
| Basic 1 peak | 9.3 | 8.2 | 5.0 | 5.8 |
| Basic 2 peak | 3.9 | 1.5 | 1.8 | ND |
| Basic variants | 3.8 | 3.7 | 4.4 | 5.0 |
| Other | ND | 0.8 | ND | ND |
| HP-SEC [%] | | | | |
| High Molecular | 0.79 | 2.34 | 4.91 | 3.68 |
| Weight Species | | | | |
| Monomer | 99.2 | 97.1 | 93.3 | 93.3 |
| Late Eluting Peaks | NQ | 0.56 | 1.74 | 3.00 |

ND-Not Detected;
NQ-Not Quantified;
RH4: 40° C./75% Relative Humidity

TABLE 7
Storage Condition 5 C.

| Lot E | Stability Test Interval (Months) | | | | | | |
|---|---|---|---|---|---|---|---|
| Test | Initial | 1 M | 3 M | 6 M | 12 M | 18 M | 24 M |
| Assay - UV (A280 nm) [mg/mL] | 94.1 | 97.5 | 97.3 | 99.0 | 91.9 | 101.3 | 98.2 |
| HP-IEX [%] | | | | | | | |
| Acidic Variants | 8.1 | 7.7 | 7.8 | 8.0 | 7.9 | 8.5 | 8.6 |
| Acidic 1 Peak | 8.3 | 8.8 | 8.8 | 8.8 | 9.1 | 9.5 | 9.6 |
| PreMain Peak | | | | | | | |
| Main peak | 66.0 | 66.0 | 66.0 | 65.8 | 65.6 | 65.2 | 65.2 |
| PosMain Peak | | | | | | | |
| Basic 1 peak | 9.5 | 9.5 | 9.7 | 9.8 | 9.9 | 9.4 | 9.4 |
| Basic 2 peak | 4.3 | 4.7 | 4.2 | 4.0 | 3.7 | 3.6 | 3.3 |
| Basic variants | 3.3 | 3.3 | 3.4 | 3.5 | 3.6 | 3.4 | 3.4 |
| Other | 0.5 | ND | ND | ND | ND | 0.5 | 0.5 |
| HP-IEX [%] | | | | | | | |
| Acidic Variants | 16.9 | NT | 17.2 | 17.8 | 18.2 | NT | 19.1 |
| Main Peak | 63.8 | NT | 63.5 | 62.7 | 63.1 | NT | 63.3 |
| Post Main Peak | 2.0 | NT | 2.1 | 2.4 | 2.1 | NT | 1.9 |
| Basic Variants | 17.3 | NT | 17.1 | 17.0 | 16.8 | NT | 15.8 |
| Binding ELISA | 112 | NT | NT | 96 | NT | NT | NT |
| Potency relative to control [%] | | | | | | | |
| Binding ELISA | NT | NT | NT | NT | 98 | 100 | 103 |
| Potency relative to control [%] | | | | | | | |
| Biological potency by Cell-based | NT | NT | NT | NT | 113 | 106 | 90 |
| functional assay | | | | | | | |
| HP-SEC [%] | | | | | | | |
| High Molecular Weight Species | 0.21 | 0.27 | 0.17 | 0.37 | 0.37 | 0.46 | 0.51 |
| Monomer | 99.8 | 99.7 | 99.8 | 99.5 | 99.5 | 99.3 | 99.3 |
| Late Eluting Peaks | NQ | 0.05 | NQ | 0.13 | <QL | <QL | <QL |
| HP-SEC [%] | | | | | | | |
| High Molecular Weight Species | NT | NT | NT | NT | NT | NT | 0.47 |
| Monomer | NT | NT | NT | NT | NT | NT | 99.2 |
| Late Eluting Peaks | NT | NT | NT | NT | NT | NT | 0.36 |
| Storage Condition | 5 C | | | | | | |
| Lot E | Stability Test Interval (Months) | | | | | | |
| HIAC [particles/mL] | | | | | | | |
| 2 μm | 1646 | 2307 | 2759 | 2240 | 5752 | 923 | 4491 |
| 5 μm | 199 | 343 | 524 | 240 | 1352 | 265 | 1733 |
| 10 μm | 15 | 96 | 136 | 30 | 275 | 48 | 585 |
| 25 μm | 0 | 3 | 4 | 1 | 15 | 0 | 11 |
| CE-SDS (Non-reduced) [%] | | | | | | | |
| Total Purity | NT | NT | NT | NT | NT | NT | 96.6 |
| CE-SDS (Reduced) [%] | | | | | | | |
| | NT | NT | NT | NT | NT | NT | 96.6 |

ND—Not Detected;
NQ—Not Quantified;
NT—Not tested;
QL—Quantification Limit;
QL = 0.25%;
5 C.: 5° C. (±3° C.)

TABLE 8
Storage Condition 25H

| Lot E | Stability Test Interval (Months) | | | | |
|---|---|---|---|---|---|
| Test | Initial | 1 M | 3 M | 6 M | 12 M |
| Assay-UV (A280 nm) | 94.1 | 98.0 | 98.2 | 96.0 | 92.9 |
| [mg/mL] | | | | | |
| HP-IEX [%] | | | | | |
| Acidic Variants | 8.1 | 9.1 | 10.9 | 14.5 | 20.0 |
| Acidic 1 Peak | 8.3 | 9.4 | 11.2 | 13.6 | 16.8 |
| Pre-Main Peak | | | | | |
| Main peak | 66.0 | 64.3 | 61.8 | 56.8 | 49.5 |
| Post-Main Peak | | | | | |
| Basic 1 peak | 9.5 | 9.6 | 9.9 | 9.5 | 7.7 |
| Basic 2 peak | 4.3 | 4.1 | 2.3 | 1.8 | 0.9 |
| Basic variants | 3.3 | 3.4 | 3.5 | 3.8 | 3.9 |
| Other | 0.5 | ND | ND | ND | 1.1 |
| Binding ELISA | 112 | NT | NT | 99 | NT |
| Potency relative | | | | | |
| to control [%] | | | | | |
| Binding ELISA | NT | NT | NT | NT | 105 |
| Potency relative | | | | | |
| to control [%] | | | | | |
| Biological potency | NT | NT | NT | NT | 103 |
| by Cell-based | | | | | |
| functional assay | | | | | |
| HP-SEC [%] | | | | | |
| High Molecular | 0.21 | 0.51 | 0.49 | 1.19 | 1.87 |
| Weight Species | | | | | |
| Monomer | 99.8 | 99.4 | 99.1 | 98.3 | 97.6 |
| Low Molecular | NQ | 0.12 | 0.43 | 0.50 | 0.86 |
| Weight Species | | | | | |

ND-Not Detected;
NT-Not tested;
NQ-Not Quantified;
25H: 25° C./60% Relative Humidity.

TABLE 9
Storage Condition RH4

| Lot E | Stability Test Interval (Months) | | | |
|---|---|---|---|---|
| Test | Initial | 1 M | 3 M | 6 M |
| Assay-UV (A280 nm) | 94.1 | 94.9 | 98.4 | 95.5 |
| [mg/mL] | | | | |
| HP-IEX [%] | | | | |
| Acidic Variants | 8.1 | 18.5 | 36.3 | 56.7 |
| Acidic 1 Peak | 8.3 | 15.1 | 19.4 | 16.5 |
| PreMain Peak | | | | |
| Main peak | 66.0 | 51.6 | 32.6 | 16.9 |
| PostMain Peak | | | | |
| Basic 1 peak | 9.5 | 8.8 | 7.3 | 5.5 |
| Basic 2 peak | 4.3 | 1.7 | 0.4 | ND |
| Basic variants | 3.3 | 3.6 | 4.1 | 4.4 |
| Other | 0.5 | 0.7 | ND | ND |
| Binding ELISA | 112 | NT | NT | 104 |
| Potency relative | | | | |
| to control [%] | | | | |
| HP-SEC [%] | | | | |
| High Molecular | 0.21 | 1.39 | 1.66 | 4.86 |
| Weight Species | | | | |
| Monomer | 99.8 | 98.1 | 96.7 | 92.7 |
| Low Molecular | NQ | 0.51 | 1.60 | 2.44 |
| Weight Species | | | | |

ND-Not Detected;
NQ-Not Quantified;
NT-Not tested;
RH4: 40° C./75% Relative Humidity

Table 10 lists the sequences in the sequence listing.

TABLE 10
Sequence Identifiers

| SEQ ID NO: | Description |
|---|---|
| 1 | huml3B8-b HC |
| 2 | huml3B8-b LC |
| 3 | 13B8-b CDRH1 |
| 4 | 13B8-b CDRH2 |
| 5 | 13B8-b CDRH3 |
| 6 | 13B8-b CDRL1 |
| 7 | 13B8-b CDRL2 |
| 8 | 13B8-b CDRL3 |
| 9 | human IL-23p19 |
| 10 | huml3B8-b HC DNA |
| 11 | huml3B8-b LC DNA |
| 12 | Heavy Chain Signal Sequence |
| 13 | Light Chain Signal Sequence |

What is claimed is:

1: A pharmaceutical formulation comprising an anti-IL-23p19 antibody hum13B8-b, wherein the anti-IL-23p19 antibody hum13B8-b comprises a light chain polypeptide having the amino acid sequence of SEQ ID NO: 2 and a heavy chain polypeptide having the amino acid sequence of SEQ ID NO: 1, and wherein the formulation comprises no more than about 25.4% acidic variants of the anti-IL-23p19 antibody hum13B8-b.

2: The pharmaceutical formulation of claim 1, wherein the formulation, after storage at about 25° C. for at least 12 months, comprises:
(a) no more than 25.4% acidic variants of the anti-IL-23p19 antibody hum13B8-b;
(b) no more than 4.2% basic variants of the anti-IL-23p19 antibody hum13B8-b;
(c) no more than 2.69% high molecular weight (HMW) species of the anti-IL-23p19 antibody hum13B8-b;
(d) no more than 0.86% low molecular weight (LMW) species of the anti-IL-23p19 antibody hum13B8-b; and
(e) at least 96.2% anti-IL-23p19 antibody hum13B8-b monomers.

3: The pharmaceutical formulation of claim 2, wherein the percentage of acid variants and/or basic variants is measured by high performance ion exchange chromatography (HP-IEX).

4: The pharmaceutical formulation of claim 2, wherein the percentage of HMW species, LMW species, and/or monomers is measured by high performance size exclusion chromatography (HP-SEC).

5: The pharmaceutical formulation of claim 1, wherein the formulation, after storage at 5°±3° C. for at least 12 months, comprises:
(a) no more than 18.2% acidic variants of the anti-IL-23p19 antibody hum13B8-b;
(b) no more than 16.8% basic variants of the anti-IL-23p19 antibody hum13B8-b;
(c) no more than 0.84% high molecular weight (HMW) species of the anti-IL-23p19 antibody hum13B8-b; and
(d) at least 98.9% anti-IL-23p19 antibody hum13B8-b monomers.

6: The pharmaceutical formulation of claim 5, wherein the percentage of acid variants and/or basic variants is measured by high performance ion exchange chromatography (HP-IEX).

7: The pharmaceutical formulation of claim 5, wherein the percentage of HMW species and/or monomers is measured by high performance size exclusion chromatography (HP-SEC).

8: The pharmaceutical formulation of claim 1, wherein the formulation comprises at least 90 to 110 mg/mL anti-IL-23p19 antibody hum13B8-b.

9: The pharmaceutical formulation of claim 8, wherein the formulation comprises at least 100 mg/mL anti-IL-23p19 antibody hum13B8-b.

10: The pharmaceutical formulation of claim 1, wherein the formulation comprises a buffer, a surfactant, and tonicity adjustment agent.

11: The pharmaceutical formulation of claim 10, wherein the buffer is histidine or acetate.

12: The pharmaceutical formulation of claim 10, wherein the surfactant is polysorbate 80, polysorbate 20, or poloxamer 188.

13: The pharmaceutical formulation of claim 10, wherein the tonicity adjustment agent is sucrose, trehlose, or mannitol.

14: The pharmaceutical formulation of claim 10, wherein the buffer is 10 mM histidine, the surfactant is 0-0.05% (w/v) of polysorbate 80, and the tonicity adjustment agent is 7% (w/v) sucrose.

15: The pharmaceutical formulation of claim 1, wherein the formulation is in a pre-filled syringe or autoinjector.

16: The pharmaceutical formulation of claim 1, wherein the formulation is for subcutaneous administration.

17: A pharmaceutical formulation comprising an anti-IL-23p19 antibody hum13B8-b, wherein the anti-IL-23p19 antibody hum13B8-b comprises a light chain polypeptide having the amino acid sequence of SEQ ID NO: 2 and a heavy chain polypeptide having the amino acid sequence of SEQ ID NO: 1, and wherein the formulation comprises no more than about 19.1% acidic variants of the anti-IL-23p19 antibody hum13B8-b.

18: The pharmaceutical formulation of claim 17, wherein the formulation, after storage at 5°±3° C. for at least 24 months, comprises:
(a) no more than 19.1% acidic variants of the anti-IL-23p19 antibody hum13B8-b;
(b) no more than 15.8% basic variants of the anti-IL-23p19 antibody hum13B8-b;
(c) no more than 1.24% high molecular weight (HMW) species of the anti-IL-23p19 antibody hum13B8-b; and
(d) at least 98.5% anti-IL-23p19 antibody hum13B8-b monomers.

19: The pharmaceutical formulation of claim 18, wherein the percentage of acid variants and/or basic variants is measured by high performance ion exchange chromatography (HP-IEX).

20: The pharmaceutical formulation of claim 18, wherein the percentage of HMW species and/or monomers is measured by high performance size exclusion chromatography (HP-SEC).

21: The pharmaceutical formulation of claim 17, wherein the formulation comprises at least 90 to 110 mg/mL anti-IL-23p19 antibody hum13B8-b.

22: The pharmaceutical formulation of claim 21, wherein the formulation comprises at least 100 mg/mL anti-IL-23p19 antibody hum13B8-b.

23: The pharmaceutical formulation of claim 17, wherein the formulation comprises a buffer, a surfactant, and tonicity adjustment agent.

24: The pharmaceutical formulation of claim 23, wherein the buffer is histidine or acetate.

25: The pharmaceutical formulation of claim 23, wherein the surfactant is polysorbate 80, polysorbate 20, or poloxamer 188.

26: The pharmaceutical formulation of claim 23, wherein the tonicity adjustment agent is sucrose, trehlose, or mannitol.

27: The pharmaceutical formulation of claim 23, wherein the buffer is 10 mM histidine, the surfactant is 0-0.05% (w/v) of polysorbate 80, and the tonicity adjustment agent is 7% (w/v) sucrose.

28: The pharmaceutical formulation of claim 17, wherein the formulation is in a pre-filled syringe or autoinjector.

29: The pharmaceutical formulation of claim 17, wherein the formulation is for subcutaneous administration.


SEQUENCE LISTING (13 sequences)

SEQ ID NO: 1 - protein, 446 aa, synthetic construct
  REGION 1..446: note = Human frameworks, rodent CDRs
  DOMAIN 1..116: note = Variable Domain
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Human frameworks, rodent CDRs
  DOMAIN 1..108: note = Variable Domain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, Mus musculus
GYIFITYWMT

SEQ ID NO: 4 - protein, 17 aa, synthetic construct
  REGION 1..17: note = Rodent CDR with one amino acid substitution
QIFPASGSADYNEKFEG

SEQ ID NO: 5 - protein, 7 aa, Mus musculus
GGGGFAY

SEQ ID NO: 6 - protein, 11 aa, Mus musculus
RTSENIYSYLA

SEQ ID NO: 7 - protein, 7 aa, Mus musculus
NAKTLAE

SEQ ID NO: 8 - protein, 9 aa, Mus musculus
QHHYGIPFT

SEQ ID NO: 9 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 10 - DNA, 1398 bp, synthetic construct
  misc_feature 1..1398: note = Human constant and framework regions, rodent CDRs
  source 1..1398: mol_type = other DNA
atggctgtgctggggctgctgttctgcctggtgacattcccaagctgtgtgctgtcccaggtgcagctggtgcagtctggcgctgaggtgaagaagcctggcgcctccgtgaaggtctcctgcaaggcttctggctacatcttcatcacctactggatgacctgggtgcggcaggcccctggccaggggctggagtggatgggccagatcttccctgccagcggctctgcagactacaacgagaagttcgaaggcagagtcaccatgaccacagacacatccaccagcacagcctacatggagctgaggagcctgagatctgacgacaccgccgtgtattactgtgccagaggcggtggcggattcgcttactggggccagggcaccctggtcaccgtctccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - DNA, 702 bp, synthetic construct
  misc_feature 1..702: note = Human constant and framework regions, rodent CDRs
  source 1..702: mol_type = other DNA
atggctccagtgcagctgctggggctgctggtgctgttcctgccagccatgagatgtgatatccagatgacccagtctccatcctccctgtctgcctctgtgggcgacagagtgaccatcacctgcaggaccagcgagaacatctacagctacctggcctggtatcagcagaagccagggaaggcccctaagctgctgatctataacgccaagaccctggctgaaggggtgccatccaggttcagcggcagcggctctgggacagacttcaccctgaccatcagcagcctgcagcctgaggacttcgccacctactactgtcagcaccactacggaattccattcaccttcggccagggcaccaaggtggagatcaagcgtacggtggctgcaccatctgtgttcatcttccctccatctgatgagcagctgaagtctggaactgcctccgtggtgtgcctgctgaataacttctatcccagagaggccaaggtgcagtggaaggtggataacgccctccagagcggcaactcccaggagagcgtgacagagcaggacagcaaggacagcacctacagcctgagcagcaccctgaccctgagcaaagcagactacgagaaacacaaggtgtacgcctgcgaggtgacccatcagggcctgagcagccccgtgacaaagagcttcaacaggggagagtgttaa

SEQ ID NO: 12 - protein, 19 aa, Mus musculus
MAVLGLLFCLVTFPSCVLS

SEQ ID NO: 13 - protein, 19 aa, Rattus norvegicus
MAPVQLLGLLVLFLPAMRC